SEQUENCE LISTING (249 sequences)

SEQ ID NO: 1 - protein, 166 aa, Homo sapiens
APPRLICDSRVLERYLLEAKEAEKITTGCAEHCSLNEKITVPDTKVNFYAWKRMEVGQQAVEVWQGLALLSEAVLRGQALLVKSSQPWEPLQLHVDKAVSGLRSLTTLLRALGAQKEAISNSDAASAAPLRTITADTFRKLFRVYSNFLRGKLKLYTGEACRTGDR

SEQ ID NO: 2 - protein, 9 aa, Artificial
  Synthetic
WSRMEVGQQ

SEQ ID NO: 3 - protein, 9 aa, Artificial
  Synthetic
WKEMEVGQQ

SEQ ID NO: 4 - protein, 9 aa, Artificial
  Synthetic
WKRMEMGQQ

SEQ ID NO: 5 - protein, 9 aa, Artificial
  Synthetic
WKRMEVGQR

SEQ ID NO: 6 - protein, 9 aa, Artificial
  Synthetic
WKRMEVGQR

SEQ ID NO: 7 - protein, 9 aa, Artificial
  Synthetic
WKRMEVGQK

SEQ ID NO: 8 - protein, 9 aa, Artificial
  Synthetic
WNRVEVGQQ

SEQ ID NO: 9 - protein, 9 aa, Artificial
  Synthetic
WNRMEAGQQ

SEQ ID NO: 10 - protein, 9 aa, Artificial
  Synthetic
WNRMELGQQ

SEQ ID NO: 11 - protein, 9 aa, Artificial
  Synthetic
WEQMEVGQQ

SEQ ID NO: 12 - protein, 9 aa, Artificial
  Synthetic
WEHMEVGQQ

SEQ ID NO: 13 - protein, 9 aa, Artificial
  Synthetic
WEKMEVGQQ

SEQ ID NO: 14 - protein, 9 aa, Artificial
  Synthetic
WERIEVGQQ

SEQ ID NO: 15 - protein, 9 aa, Artificial
  Synthetic
WERLEVGQQ

SEQ ID NO: 16 - protein, 9 aa, Artificial
  Synthetic
WERVEVGQQ

SEQ ID NO: 17 - protein, 9 aa, Artificial
  Synthetic
WERFEVGQQ

SEQ ID NO: 18 - protein, 9 aa, Artificial
  Synthetic
WERMEAGQQ

SEQ ID NO: 19 - protein, 9 aa, Artificial
  Synthetic
WERMEIGQQ

SEQ ID NO: 20 - protein, 9 aa, Artificial
  Synthetic
WERMELGQQ

SEQ ID NO: 21 - protein, 9 aa, Artificial
  Synthetic
WERMEVGQS

SEQ ID NO: 22 - protein, 9 aa, Artificial
  Synthetic
WERMEVGQN

SEQ ID NO: 23 - protein, 9 aa, Artificial
  Synthetic
WERMEVGQD

SEQ ID NO: 24 - protein, 9 aa, Artificial
  Synthetic
WERMEVGQM

SEQ ID NO: 25 - protein, 9 aa, Artificial
  Synthetic
WKNVEVGQQ

SEQ ID NO: 26 - protein, 9 aa, Artificial
  Synthetic
WKNMEAGQQ

SEQ ID NO: 27 - protein, 9 aa, Artificial
  Synthetic
WKQIEVGQQ

SEQ ID NO: 28 - protein, 9 aa, Artificial
  Synthetic
WKQVEVGQQ

SEQ ID NO: 29 - protein, 9 aa, Artificial
  Synthetic
WKQFEVGQQ

SEQ ID NO: 30 - protein, 9 aa, Artificial
  Synthetic
WKQMEAGQQ

SEQ ID NO: 31 - protein, 9 aa, Artificial
  Synthetic
WKQMELGQQ

SEQ ID NO: 32 - protein, 9 aa, Artificial
  Synthetic
WKQMEVGQN

SEQ ID NO: 33 - protein, 9 aa, Artificial
  Synthetic
WKQMEVGQM

SEQ ID NO: 34 - protein, 9 aa, Artificial
  Synthetic
WKHVEVGQQ

SEQ ID NO: 35 - protein, 9 aa, Artificial
  Synthetic
WKHFEVGQQ

SEQ ID NO: 36 - protein, 9 aa, Artificial
  Synthetic
WKHMEAGQQ

SEQ ID NO: 37 - protein, 9 aa, Artificial
  Synthetic
WKHMELGQQ

SEQ ID NO: 38 - protein, 9 aa, Artificial
  Synthetic
WKKIEVGQQ

SEQ ID NO: 39 - protein, 9 aa, Artificial
  Synthetic
WKKVEVGQQ

SEQ ID NO: 40 - protein, 9 aa, Artificial
  Synthetic
WKKFEVGQQ

SEQ ID NO: 41 - protein, 9 aa, Artificial
  Synthetic
WKKMEAGQQ

SEQ ID NO: 42 - protein, 9 aa, Artificial
  Synthetic
WKKMELGQQ

SEQ ID NO: 43 - protein, 9 aa, Artificial
  Synthetic
WKKMEVGQN

SEQ ID NO: 44 - protein, 9 aa, Artificial
  Synthetic
WKKMEVGQM

SEQ ID NO: 45 - protein, 9 aa, Artificial
  Synthetic
WKRIEAGQQ

SEQ ID NO: 46 - protein, 9 aa, Artificial
  Synthetic
WKRIELGQQ

SEQ ID NO: 47 - protein, 9 aa, Artificial
  Synthetic
WKRIEVGQN

SEQ ID NO: 48 - protein, 9 aa, Artificial
  Synthetic
WKRIEVGQD

SEQ ID NO: 49 - protein, 9 aa, Artificial
  Synthetic
WKRIEVGQM

SEQ ID NO: 50 - protein, 9 aa, Artificial
  Synthetic
WKRLEAGQQ

SEQ ID NO: 51 - protein, 9 aa, Artificial
  Synthetic
WKRLELGQQ

SEQ ID NO: 52 - protein, 9 aa, Artificial
  Synthetic
WKRLEVGQN

SEQ ID NO: 53 - protein, 9 aa, Artificial
  Synthetic
WKRLEVGQM

SEQ ID NO: 54 - protein, 9 aa, Artificial
  Synthetic
WKRVEAGQQ

SEQ ID NO: 55 - protein, 9 aa, Artificial
  Synthetic
WKRVEIGQQ

SEQ ID NO: 56 - protein, 9 aa, Artificial
  Synthetic
WKRVELGQQ

SEQ ID NO: 57 - protein, 9 aa, Artificial
  Synthetic
WKRVEVGQN

SEQ ID NO: 58 - protein, 9 aa, Artificial
  Synthetic
WKRVEVGQD

SEQ ID NO: 59 - protein, 9 aa, Artificial
  Synthetic
WKRVEVGQM

SEQ ID NO: 60 - protein, 9 aa, Artificial
  Synthetic
WKRFEAGQQ

SEQ ID NO: 61 - protein, 9 aa, Artificial
  Synthetic
WKRFEIGQQ

SEQ ID NO: 62 - protein, 9 aa, Artificial
  Synthetic
WKRFELGQQ

SEQ ID NO: 63 - protein, 9 aa, Artificial
  Synthetic
WKRFEVGQN

SEQ ID NO: 64 - protein, 9 aa, Artificial
  Synthetic
WKRFEVGQM

SEQ ID NO: 65 - protein, 9 aa, Artificial
  Synthetic
WKRMEAGQS

SEQ ID NO: 66 - protein, 9 aa, Artificial
  Synthetic
WKRMEAGQN

SEQ ID NO: 67 - protein, 9 aa, Artificial
  Synthetic
WKRMEAGQD

SEQ ID NO: 68 - protein, 9 aa, Artificial
  Synthetic
WKRMEAGQM

SEQ ID NO: 69 - protein, 9 aa, Artificial
  Synthetic
WKRMELGQS

SEQ ID NO: 70 - protein, 9 aa, Artificial
  Synthetic
WKRMELGQN

SEQ ID NO: 71 - protein, 9 aa, Artificial
  Synthetic
WKRMELGQD

SEQ ID NO: 72 - protein, 9 aa, Artificial
  Synthetic
WKRMELGQM

SEQ ID NO: 73 - protein, 9 aa, Artificial
  Synthetic
WNQMEIGQQ

SEQ ID NO: 74 - protein, 9 aa, Artificial
  Synthetic
WNKMEIGQQ

SEQ ID NO: 75 - protein, 9 aa, Artificial
  Synthetic
WNRIEIGQQ

SEQ ID NO: 76 - protein, 9 aa, Artificial
  Synthetic
WKNIEIGQQ

SEQ ID NO: 77 - protein, 9 aa, Artificial
  Synthetic
WKQLEIGQQ

SEQ ID NO: 78 - protein, 9 aa, Artificial
  Synthetic
WKQLEVGQE

SEQ ID NO: 79 - protein, 9 aa, Artificial
  Synthetic
WKQMEIGQS

SEQ ID NO: 80 - protein, 9 aa, Artificial
  Synthetic
WKQMEIGQD

SEQ ID NO: 81 - protein, 9 aa, Artificial
  Synthetic
WKHIEIGQQ

SEQ ID NO: 82 - protein, 9 aa, Artificial
  Synthetic
WKHLEIGQQ

SEQ ID NO: 83 - protein, 9 aa, Artificial
  Synthetic
WKKLEIGQQ

SEQ ID NO: 84 - protein, 9 aa, Artificial
  Synthetic
WKKLEVGQE

SEQ ID NO: 85 - protein, 9 aa, Artificial
  Synthetic
WKKMEIGQS

SEQ ID NO: 86 - protein, 9 aa, Artificial
  Synthetic
WKKMEIGQD

SEQ ID NO: 87 - protein, 9 aa, Artificial
  Synthetic
WKRIEIGQE

SEQ ID NO: 88 - protein, 9 aa, Artificial
  Synthetic
WKRIEIGQH

SEQ ID NO: 89 - protein, 9 aa, Artificial
  Synthetic
WKRLEIGQD

SEQ ID NO: 90 - protein, 9 aa, Artificial
  Synthetic
LNSLTTLLR

SEQ ID NO: 91 - protein, 9 aa, Artificial
  Synthetic
LESLTTLLR

SEQ ID NO: 92 - protein, 9 aa, Artificial
  Synthetic
LQSLTTLLR

SEQ ID NO: 93 - protein, 9 aa, Artificial
  Synthetic
LHSLTTLLR

SEQ ID NO: 94 - protein, 9 aa, Artificial
  Synthetic
LKSLTTLLR

SEQ ID NO: 95 - protein, 9 aa, Artificial
  Synthetic
LRDLTTLLR

SEQ ID NO: 96 - protein, 9 aa, Artificial
  Synthetic
LRELTTLLR

SEQ ID NO: 97 - protein, 9 aa, Artificial
  Synthetic
LRTLTSLLR

SEQ ID NO: 98 - protein, 9 aa, Artificial
  Synthetic
LRTLTALLR

SEQ ID NO: 99 - protein, 9 aa, Artificial
  Synthetic
LRALTSLLR

SEQ ID NO: 100 - protein, 9 aa, Artificial
  Synthetic
LRALTALLR

SEQ ID NO: 101 - protein, 9 aa, Artificial
  Synthetic
LRQLTSLLR

SEQ ID NO: 102 - protein, 9 aa, Artificial
  Synthetic
LRKLTSLLR

SEQ ID NO: 103 - protein, 9 aa, Artificial
  Synthetic
LRSITALLR

SEQ ID NO: 104 - protein, 9 aa, Artificial
  Synthetic
LRSVTSLLR

SEQ ID NO: 105 - protein, 9 aa, Artificial
  Synthetic
LRSVTALLR

SEQ ID NO: 106 - protein, 9 aa, Artificial
  Synthetic
LRSVTNLLR

SEQ ID NO: 107 - protein, 9 aa, Artificial
  Synthetic
LRSVTTVLR

SEQ ID NO: 108 - protein, 9 aa, Artificial
  Synthetic
LRSVTTFLR

SEQ ID NO: 109 - protein, 9 aa, Artificial
  Synthetic
LRSLTSILR

SEQ ID NO: 110 - protein, 9 aa, Artificial
  Synthetic
LRSLTSVLR

SEQ ID NO: 111 - protein, 9 aa, Artificial
  Synthetic
LRSLTSFLR

SEQ ID NO: 112 - protein, 9 aa, Artificial
  Synthetic
LRSLTAMLR

SEQ ID NO: 113 - protein, 9 aa, Artificial
  Synthetic
LRSLTNILR

SEQ ID NO: 114 - protein, 9 aa, Artificial
  Synthetic
LRSLTNVLR

SEQ ID NO: 115 - protein, 9 aa, Artificial
  Synthetic
LRTVTTILR

SEQ ID NO: 116 - protein, 9 aa, Artificial
  Synthetic
LRAVTTILR

SEQ ID NO: 117 - protein, 9 aa, Artificial
  Synthetic
FNKLFRVYS

SEQ ID NO: 118 - protein, 9 aa, Artificial
  Synthetic
FEKLFRVYS

SEQ ID NO: 119 - protein, 9 aa, Artificial
  Synthetic
FQKLFRVYS

SEQ ID NO: 120 - protein, 9 aa, Artificial
  Synthetic
FHKLFRVYS

SEQ ID NO: 121 - protein, 9 aa, Artificial
  Synthetic
FKKLFRVYS

SEQ ID NO: 122 - protein, 9 aa, Artificial
  Synthetic
FRELFRVYS

SEQ ID NO: 123 - protein, 9 aa, Artificial
  Synthetic
FRKLFEVYS

SEQ ID NO: 124 - protein, 9 aa, Artificial
  Synthetic
FRKLFQVYS

SEQ ID NO: 125 - protein, 9 aa, Artificial
  Synthetic
FRKLFHVYS

SEQ ID NO: 126 - protein, 9 aa, Artificial
  Synthetic
FRKLFRVYT

SEQ ID NO: 127 - protein, 9 aa, Artificial
  Synthetic
FRKLFRVYN

SEQ ID NO: 128 - protein, 9 aa, Artificial
  Synthetic
FRKLFRVYD

SEQ ID NO: 129 - protein, 9 aa, Artificial
  Synthetic
MRKVFRVYS

SEQ ID NO: 130 - protein, 9 aa, Artificial
  Synthetic
MRKLFRTYS

SEQ ID NO: 131 - protein, 9 aa, Artificial
  Synthetic
MRKLFRVYA

SEQ ID NO: 132 - protein, 9 aa, Artificial
  Synthetic
IRKVFRVYS

SEQ ID NO: 133 - protein, 9 aa, Artificial
  Synthetic
IRKLFRTYS

SEQ ID NO: 134 - protein, 9 aa, Artificial
  Synthetic
IRKLFRVYA

SEQ ID NO: 135 - protein, 9 aa, Artificial
  Synthetic
LRKVFRVYS

SEQ ID NO: 136 - protein, 9 aa, Artificial
  Synthetic
LRKLFRTYS

SEQ ID NO: 137 - protein, 9 aa, Artificial
  Synthetic
LRKLFRVYA

SEQ ID NO: 138 - protein, 9 aa, Artificial
  Synthetic
FRKMFRVYK

SEQ ID NO: 139 - protein, 9 aa, Artificial
  Synthetic
FRKIFRVYG

SEQ ID NO: 140 - protein, 9 aa, Artificial
  Synthetic
FRKIFRVYE

SEQ ID NO: 141 - protein, 9 aa, Artificial
  Synthetic
FRKIFRVYK

SEQ ID NO: 142 - protein, 9 aa, Artificial
  Synthetic
FRKVFRTYS

SEQ ID NO: 143 - protein, 9 aa, Artificial
  Synthetic
FRKVFRVYA

SEQ ID NO: 144 - protein, 9 aa, Artificial
  Synthetic
FRKVFRVYG

SEQ ID NO: 145 - protein, 9 aa, Artificial
  Synthetic
FRKVFRVYE

SEQ ID NO: 146 - protein, 9 aa, Artificial
  Synthetic
FRKVFRVYK

SEQ ID NO: 147 - protein, 9 aa, Artificial
  Synthetic
FRKFFRVYE

SEQ ID NO: 148 - protein, 9 aa, Artificial
  Synthetic
FRKFFRVYK

SEQ ID NO: 149 - protein, 9 aa, Artificial
  Synthetic
FRKLFRTYA

SEQ ID NO: 150 - protein, 9 aa, Artificial
  Synthetic
FRKLFRTYG

SEQ ID NO: 151 - protein, 9 aa, Artificial
  Synthetic
FRKLFRTYE

SEQ ID NO: 152 - protein, 9 aa, Artificial
  Synthetic
FRKLFRTYK

SEQ ID NO: 153 - protein, 9 aa, Artificial
  Synthetic
FRKIFRTYQ

SEQ ID NO: 154 - protein, 9 aa, Artificial
  Synthetic
LWRVYSNFL

SEQ ID NO: 155 - protein, 9 aa, Artificial
  Synthetic
LFEVYSNFL

SEQ ID NO: 156 - protein, 9 aa, Artificial
  Synthetic
LFQVYSNFL

SEQ ID NO: 157 - protein, 9 aa, Artificial
  Synthetic
LFHVYSNFL

SEQ ID NO: 158 - protein, 9 aa, Artificial
  Synthetic
LFKVYSNFL

SEQ ID NO: 159 - protein, 9 aa, Artificial
  Synthetic
LFRAYSNFL

SEQ ID NO: 160 - protein, 9 aa, Artificial
  Synthetic
LFRLYSNFL

SEQ ID NO: 161 - protein, 9 aa, Artificial
  Synthetic
LFRVYTNFL

SEQ ID NO: 162 - protein, 9 aa, Artificial
  Synthetic
LFRVYANFL

SEQ ID NO: 163 - protein, 9 aa, Artificial
  Synthetic
LFRVYGNFL

SEQ ID NO: 164 - protein, 9 aa, Artificial
  Synthetic
LFRVYNNFL

SEQ ID NO: 165 - protein, 9 aa, Artificial
  Synthetic
LFRVYDNFL

SEQ ID NO: 166 - protein, 9 aa, Artificial
  Synthetic
LFRVYENFL

SEQ ID NO: 167 - protein, 9 aa, Artificial
  Synthetic
LFRVYQNFL

SEQ ID NO: 168 - protein, 9 aa, Artificial
  Synthetic
LFRVYKNFL

SEQ ID NO: 169 - protein, 9 aa, Artificial
  Synthetic
LFRVYSSFL

SEQ ID NO: 170 - protein, 9 aa, Artificial
  Synthetic
LFRVYSTFL

SEQ ID NO: 171 - protein, 9 aa, Artificial
  Synthetic
LFRVYSGFL

SEQ ID NO: 172 - protein, 9 aa, Artificial
  Synthetic
LFRVYSDFL

SEQ ID NO: 173 - protein, 9 aa, Artificial
  Synthetic
LFRVYSEFL

SEQ ID NO: 174 - protein, 9 aa, Artificial
  Synthetic
LFRVYSQFL

SEQ ID NO: 175 - protein, 9 aa, Artificial
  Synthetic
LFRVYSHFL

SEQ ID NO: 176 - protein, 9 aa, Artificial
  Synthetic
LFRVYSRFL

SEQ ID NO: 177 - protein, 9 aa, Artificial
  Synthetic
LFRVYSKFL

SEQ ID NO: 178 - protein, 9 aa, Artificial
  Synthetic
LFRVYSNFM

SEQ ID NO: 179 - protein, 9 aa, Artificial
  Synthetic
LFRVYSNFV

SEQ ID NO: 180 - protein, 9 aa, Artificial
  Synthetic
LFRVYSNFF

SEQ ID NO: 181 - protein, 9 aa, Artificial
  Synthetic
FEVYSNFLR

SEQ ID NO: 182 - protein, 9 aa, Artificial
  Synthetic
FRVYSEFLR

SEQ ID NO: 183 - protein, 9 aa, Artificial
  Synthetic
FNTYSNFLR

SEQ ID NO: 184 - protein, 9 aa, Artificial
  Synthetic
FNAYSNFLR

SEQ ID NO: 185 - protein, 9 aa, Artificial
  Synthetic
FNVYSNYLR

SEQ ID NO: 186 - protein, 9 aa, Artificial
  Synthetic
FNVYSNFLQ

SEQ ID NO: 187 - protein, 9 aa, Artificial
  Synthetic
FNVYSNFLK

SEQ ID NO: 188 - protein, 9 aa, Artificial
  Synthetic
FQVHSNFLR

SEQ ID NO: 189 - protein, 9 aa, Artificial
  Synthetic
FQVWSNFLR

SEQ ID NO: 190 - protein, 9 aa, Artificial
  Synthetic
FQVYSDFLR

SEQ ID NO: 191 - protein, 9 aa, Artificial
  Synthetic
FQVYSHFLR

SEQ ID NO: 192 - protein, 9 aa, Artificial
  Synthetic
FQVYSNYLR

SEQ ID NO: 193 - protein, 9 aa, Artificial
  Synthetic
FQVYSNWLR

SEQ ID NO: 194 - protein, 9 aa, Artificial
  Synthetic
FQVYSNFLE

SEQ ID NO: 195 - protein, 9 aa, Artificial
  Synthetic
FQVYSNFLK

SEQ ID NO: 196 - protein, 9 aa, Artificial
  Synthetic
FHTYSNFLR

SEQ ID NO: 197 - protein, 9 aa, Artificial
  Synthetic
FHAYSNFLR

SEQ ID NO: 198 - protein, 9 aa, Artificial
  Synthetic
FHVYSNYLR

SEQ ID NO: 199 - protein, 9 aa, Artificial
  Synthetic
FHVYSNFLQ

SEQ ID NO: 200 - protein, 9 aa, Artificial
  Synthetic
FHVYSNFLK

SEQ ID NO: 201 - protein, 9 aa, Artificial
  Synthetic
FKTYSNFLR

SEQ ID NO: 202 - protein, 9 aa, Artificial
  Synthetic
FKAYSNFLR

SEQ ID NO: 203 - protein, 9 aa, Artificial
  Synthetic
FKVHSNFLR

SEQ ID NO: 204 - protein, 9 aa, Artificial
  Synthetic
FKVWSNFLR

SEQ ID NO: 205 - protein, 9 aa, Artificial
  Synthetic
FKVYSDFLR

SEQ ID NO: 206 - protein, 9 aa, Artificial
  Synthetic
FKVYSQFLR

SEQ ID NO: 207 - protein, 9 aa, Artificial
  Synthetic
FKVYSHFLR

SEQ ID NO: 208 - protein, 9 aa, Artificial
  Synthetic
FKVYSNYLR

SEQ ID NO: 209 - protein, 9 aa, Artificial
  Synthetic
FKVYSNWLR

SEQ ID NO: 210 - protein, 9 aa, Artificial
  Synthetic
FKVYSNFLE

SEQ ID NO: 211 - protein, 9 aa, Artificial
  Synthetic
FKVYSNFLQ

SEQ ID NO: 212 - protein, 9 aa, Artificial
  Synthetic
FKVYSNFLK

SEQ ID NO: 213 - protein, 9 aa, Artificial
  Synthetic
FRTWSNFLR

SEQ ID NO: 214 - protein, 9 aa, Artificial
  Synthetic
FRTYSDFLR

SEQ ID NO: 215 - protein, 9 aa, Artificial
  Synthetic
FRTYSQFLR

SEQ ID NO: 216 - protein, 9 aa, Artificial
  Synthetic
FRTYSHFLR

SEQ ID NO: 217 - protein, 9 aa, Artificial
  Synthetic
FRAWSNFLR

SEQ ID NO: 218 - protein, 9 aa, Artificial
  Synthetic
FRAYSDFLR

SEQ ID NO: 219 - protein, 9 aa, Artificial
  Synthetic
FRAYSQFLR

SEQ ID NO: 220 - protein, 9 aa, Artificial
  Synthetic
FRAYSHFLR

SEQ ID NO: 221 - protein, 9 aa, Artificial
  Synthetic
FRVYSDYLR

SEQ ID NO: 222 - protein, 9 aa, Artificial
  Synthetic
FRVYSDFLQ

SEQ ID NO: 223 - protein, 9 aa, Artificial
  Synthetic
FRVYSDFLK

SEQ ID NO: 224 - protein, 9 aa, Artificial
  Synthetic
FRVYSQYLR

SEQ ID NO: 225 - protein, 9 aa, Artificial
  Synthetic
FRVYSQFLK

SEQ ID NO: 226 - protein, 9 aa, Artificial
  Synthetic
FRVYSHYLR

SEQ ID NO: 227 - protein, 9 aa, Artificial
  Synthetic
FRVYSHFLK

SEQ ID NO: 228 - protein, 9 aa, Artificial
  Synthetic
FRTYSNYLK

SEQ ID NO: 229 - protein, 9 aa, Artificial
  Synthetic
FRAYSNYLK

SEQ ID NO: 230 - protein, 9 aa, Artificial
  Synthetic
LNGKLKLYT

SEQ ID NO: 231 - protein, 9 aa, Artificial
  Synthetic
LEGKLKLYT

SEQ ID NO: 232 - protein, 9 aa, Artificial
  Synthetic
LQGKLKLYT

SEQ ID NO: 233 - protein, 9 aa, Artificial
  Synthetic
LHGKLKLYT

SEQ ID NO: 234 - protein, 9 aa, Artificial
  Synthetic
LKGKLKLYT

SEQ ID NO: 235 - protein, 9 aa, Artificial
  Synthetic
LRGSLKLYT

SEQ ID NO: 236 - protein, 9 aa, Artificial
  Synthetic
LRGELKLYT

SEQ ID NO: 237 - protein, 9 aa, Artificial
  Synthetic
LRGQLKLYT

SEQ ID NO: 238 - protein, 9 aa, Artificial
  Synthetic
LRGKLSLYT

SEQ ID NO: 239 - protein, 9 aa, Artificial
  Synthetic
LRGKLNLYT

SEQ ID NO: 240 - protein, 9 aa, Artificial
  Synthetic
LRGKLELYT

SEQ ID NO: 241 - protein, 9 aa, Artificial
  Synthetic
LRGKLQLYT

SEQ ID NO: 242 - protein, 9 aa, Artificial
  Synthetic
LRGKLKIYT

SEQ ID NO: 243 - protein, 9 aa, Artificial
  Synthetic
LRGKLKVYT

SEQ ID NO: 244 - protein, 9 aa, Artificial
  Synthetic
LRAKLRLYT

SEQ ID NO: 245 - protein, 9 aa, Artificial
  Synthetic
LRGNLRLYT

SEQ ID NO: 246 - protein, 9 aa, Artificial
  Synthetic
LRGNLKMYT

SEQ ID NO: 247 - protein, 9 aa, Artificial
  Synthetic
LRGNLKFYT

SEQ ID NO: 248 - protein, 9 aa, Artificial
  Synthetic
LRGRLKFYT

SEQ ID NO: 249 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

(19) United States
(12) Patent Application Publication
(10) Pub. No.: US 2006/0073563 A1
(43) Pub. Date: Apr. 6, 2006

(54) Erythropoietin derivatives with altered immunogenicity

(75) Inventor: Shannon Alicia Marshall, San Francisco, CA (US)

Correspondence Address: DORSEY & WHITNEY LLP, 555 CALIFORNIA STREET, SUITE 1000, SUITE 1000, SAN FRANCISCO, CA, 94104, US

(73) Assignee: Xencor, Inc., Monrovia, CA (US)

(21) Appl. No.: 11/218,928

(22) Filed: Sep. 2, 2005

Related U.S. Application Data
(60) Provisional application No. 60/607,461, filed on Sep. 2, 2004.

(51) Int. Cl. C07H 21/04; C12P 21/06; C07K 14/505

(52) U.S. Cl. 435/69.1; 435/320.1; 435/325; 530/399; 536/23.5

(57) ABSTRACT

The present invention relates to novel erythropoietin protein variants with altered immunogenicity.

This application claims benefit under 35 USC 119(e) to U.S. Provisional Application No. 60/607,461 filed Sep. 2, 2004, which is incorporated by reference in its entirety.

### FIELD OF THE INVENTION

The present invention relates to variant erythropoietin (EPO) proteins with reduced immunogenicity. In particular, variants of EPO with reduced ability to bind one or more human class II MHC molecules are described.

### BACKGROUND OF THE INVENTION

Erythropoietin or EPO is well known and characterized in the art for possessing erythropoiesis stimulating activity. See, for example, U.S. Pat. Nos. 4,667,195 and 4,703,008, both incorporated entirely by reference. Erythropoietin EPO is an acidic glycoprotein hormone of about 34 kD molecular weight. It is comprised of 165 amino acids and has four carbohydrate chains (three N-linked and one O-linked), which have been shown to affect the protein's stability, solubility, and in vivo bioactivity but are not required for receptor binding. It is a member of the cytokine family that includes interleukins 2-7, G-CSF, GM-CSF, TPO, growth hormone and leptin. EPO induces proliferation and differentiation of erythroid progenitor cells into erythrocytes, stimulates hemoglobin C synthesis, and increases hematocrit levels. Stimulation of erythropoiesis involves the binding of EPO to the extracellular domain of the EPO receptor (EPOR) on the surface of erythroid progenitor cells; this association triggers intracellular signaling events including phosphorylation of the receptor and activation of the JAK-STAT, RAS and P13 kinase pathways. These signaling pathways trigger cells to undergo proliferation and differentiation and to prevent apoptosis. EPO is produced by the liver in the fetus and by the kidney in adults; it circulates in the blood to stimulate production of red blood cells in bone marrow. Anemia is almost invariably a consequence of renal failure due to decreased production of EPO from the kidney.

EPO receptors are also found in numerous nonerythroid cells including myeloid cells, lymphocytes, megakaryocytes, endothelial cells, mesangial, myocardial, and smooth muscle cells, as well as neural, prostate, and renal cells. Many of these cell types have active EPO signaling pathways and biologic responses. EPO has been shown to be important in the development of new blood vessels and has biologic effects in many tissues especially in the brain, ovary, oviduct, uterus, and testis, as well as in selected tumors. See Weiss Oncologist 8 Suppl 3: 18-29 (2003), incorporated entirely by reference.

Recombinant human EPO (rHuEPO) is in clinical use worldwide for the treatment of anemias deriving from renal failure, chemotherapy, and AIDS. It is also used to treat anemia in premature infants, to reduce the need for blood transfusions due to trauma or surgery, and to treat renal disease and patients undergoing dialysis. Other indications include use in autologous blood transfusions, refractory anemia, and anemia associated with hematological malignancies and other blood disorders such as hemophilia and sickle cell disease. See, for example, U.S. Pat. Nos. 4,667,195 and 4,703,008. A rHuEPO dimer has also been investigated as a potential therapy for immune disorders and for hematopoietic restoration following radio- or chemotherapy; the dimer was found to be more active than the monomeric form in vitro and in vivo (see Sytkowski et al. Proc Natl Acad Sci USA 95: 1184-1188 (1998), incorporated entirely by reference).

EPO also has important neuroprotective effects. EPO and EpoR are abundant in the brain and spinal cord and are significantly upregulated by metabolic stress. In rodent models, rHuEPO was found to cross the blood brain barrier and attenuate ischemia-induced inflammation by reducing neuronal death, indicating its potential utility in the treatment of cerebral ischemia and brain trauma (Kalialis and Olsen Ugeskr Laeger 165: 2477-2481 (2003), Villa et al. J Exp Med 198: 971-975 (2003), both incorporated entirely by reference). Studies in a rat model of experimental autoimmune encephalomyelitis (EAE) indicate that EPO may act as a protective cytokine in inflammatory pathologies of the CNS (Agnello et al. Brain Res 952: 128-134 (2002), incorporated entirely by reference). Epo's neuroprotective effects have also been exhibited in other preclinical models of CNS disorders including multiple sclerosis, spinal cord trauma, and light- or ischemia-induced retinal damage. In initial clinical studies, rHuEPO appeared to reduce the damage from stroke (Ehrenreich et al. Mol Med 8: 495-505 (2002), incorporated entirely by reference). Possible mechanisms of EPO's neuroprotective effects include prevention of glutamate-induced toxicity, inhibition of apoptosis, anti-inflammatory effects, antioxidant effects, and stimulation of angiogenesis.

Immunogenicity of EPO

Immunogenicity is a major barrier to the development and utilization of protein therapeutics. Although immune responses are typically most severe for non-human proteins, even therapeutics based on human proteins may be immunogenic. Immunogenicity is a complex series of responses to a substance that is perceived as foreign and may include production of neutralizing and non-neutralizing antibodies, formation of immune complexes, complement activation, mast cell activation, inflammation, and anaphylaxis.

Several factors can contribute to protein immunogenicity, including but not limited to the protein sequence, the route and frequency of administration, and the patient population.

Immunogenicity may limit the efficacy and safety of a protein therapeutic in multiple ways. Efficacy can be reduced directly by the formation of neutralizing antibodies. Efficacy may also be reduced indirectly, as binding to either neutralizing or non-neutralizing antibodies typically leads to rapid clearance from serum. Severe side effects and even death may occur when an immune reaction is raised. One special class of side effects results when neutralizing antibodies cross-react with an endogenous protein and block its function.

Incidence of immunogenic response to rHuEPO is rare (<1:10,000 patient-years). However, since 1998, more than 200 patients with chronic kidney disease treated with rHuEPO developed cross-reactive neutralizing antibodies (to endogenous Epo), causing pure red cell aplasia (PRCA). All of these patients were receiving EPO subcutaneously, and the product most typically prescribed was epoetin alfa (Eprex®, Ortho Biotech). It has been suggested that since the rates of PRCA remained low for other EPO products (Epogen® and NeoRecorman®), the increase in PRCA was likely product-specific. Possible mechanisms that have been identified include modification of drug formulation and down stream processing. See Casadevall et al. N Engl J Med 346: 469-475 (2002), Macdougall Curr Med Res Opin 20: 83-86 (2004), Verhelst et al. Lancet 363: 1768-1771 (2004), Locatelli and Del Vecchio J Nephrol 16: 461-466 (2003), all incorporated entirely by reference.

In an attempt to decrease aggregation of E coli-derived EPO, investigators replaced N-glycosylation site residues N24, N38, and N83 with basic residues (lysines) and improved the stability of the protein (Narhi et al. Protein Eng 14: 135-140 (2001), incorporated entirely by reference). Although these studies were done to facilitate high-resolution structure analysis, decreasing the formation of aggregates is also likely to reduce immunogenicity.

Several methods have been developed to modulate the immunogenicity of proteins. In some cases, PEGylation has been observed to reduce the fraction of patients who raise neutralizing antibodies by sterically blocking access to antibody agretopes (see for example, Hershfield et al. PNAS 1991 88:7185-7189 (1991); Bailon. et al. Bioconjug. Chem. 12: 195-202(2001); He et al. Life Sci. 65: 355-368 (1999), all incorporated entirely by reference). Methods that improve the solution properties of a protein therapeutic may also reduce immunogenicity, as aggregates have been observed to be more immunogenic than soluble proteins.

A more general approach to immunogenicity reduction involves mutagenesis targeted at the agretopes in the protein sequence and structure that are most responsible for stimulating the immune system. Some success has been achieved by randomly replacing solvent-exposed residues to lower binding affinity to panels of known neutralizing antibodies (see for example Laroche et al. Blood 96: 1425-1432 (2000), incorporated entirely by reference). Due to the incredible diversity of the antibody repertoire, mutations that lower affinity to known antibodies will typically lead to production of an another set of antibodies rather than abrogation of immunogenicity. However, in some cases it may be possible to decrease surface antigenicity by replacing hydrophobic and charged residues on the protein surface with polar neutral residues (see Meyer et al. Protein Sci. 10: 491-503 (2001), incorporated entirely by reference).

An alternate approach is to disrupt T-cell activation. Removal of MHC-binding agretopes offers a much more tractable approach to immunogenicity reduction, as the diversity of MHC molecules comprises only ˜10³ alleles, while the antibody repertoire is estimated to be approximately 10⁸ and the T-cell receptor repertoire is larger still. By identifying and removing or modifying class II MHC-binding peptides within a protein sequence, the molecular basis of immunogenicity can be evaded. The elimination of such agretopes for the purpose of generating less immunogenic proteins has been disclosed previously; see for example WO 98/52976, WO 02/079232, and WO 00/3317, all incorporated entirely by reference. Examples of replacing MHC binding agretopes in EPO have also been disclosed; see WO 02/062843, incorporated entirely by reference.

While mutations in MHC-binding agretopes can be identified that are predicted to confer reduced immunogenicity, most amino acid substitutions are energetically unfavorable. As a result, the vast majority of the reduced immunogenicity sequences identified using the methods described above will be incompatible with the structure and/or function of the protein. In order for MHC agretope removal to be a viable approach for reducing immunogenicity, it is crucial that simultaneous efforts are made to maintain a protein's structure, stability, and biological activity.

There remains a need for novel EPO proteins having reduced immunogenicity. Variants of EPO with reduced immunogenicity could find use in the treatment of a number of EPO responsive conditions.

### SUMMARY OF THE INVENTION

In accordance with the objects outlined above, the present invention provides novel erythropoietin proteins having reduced immunogenicity as compared to naturally occurring EPO proteins. In an additional aspect, the present invention is directed to methods for engineering or designing less immunogenic proteins with EPO activity for therapeutic use.

An aspect of the present invention are EPO variants that show decreased binding affinity for one or more class II MHC alleles relative to a parent EPO and which significantly maintain the activity of native naturally occurring Epo.

In a further aspect, the invention provides recombinant nucleic acids encoding the variant EPO proteins, expression vectors, and host cells.

In an additional aspect, the invention provides methods of producing a variant EPO protein comprising culturing the host cells of the invention under conditions suitable for expression of the variant EPO protein.

In a further aspect, the invention provides pharmaceutical compositions comprising a variant EPO protein or nucleic acid of the invention and a pharmaceutical carrier.

In a further aspect, the invention provides methods for preventing or treating EPO responsive disorders comprising administering a variant EPO protein or nucleic acid of the invention to a patient.

In an additional aspect, the invention provides methods for screening the class II MHC haplotypes of potential patients in order to identify individuals who are particularly likely to raise an immune response to a wild type or variant EPO therapeutic.

In accordance with the objects outlined above, the present invention provides EPO variant proteins comprising amino acid sequences with at least one amino acid insertion, deletion, or substitution compared to the wild type EPO proteins.

### BRIEF DESCRIPTION OF THE FIGURES

FIG. 1 shows a method for engineering less immunogenic erythropoietin derivatives.

FIG. 2 shows a schematic representation of a method for in vitro testing of the immunogenicity of erythropoietin peptides or proteins with IVV technology.

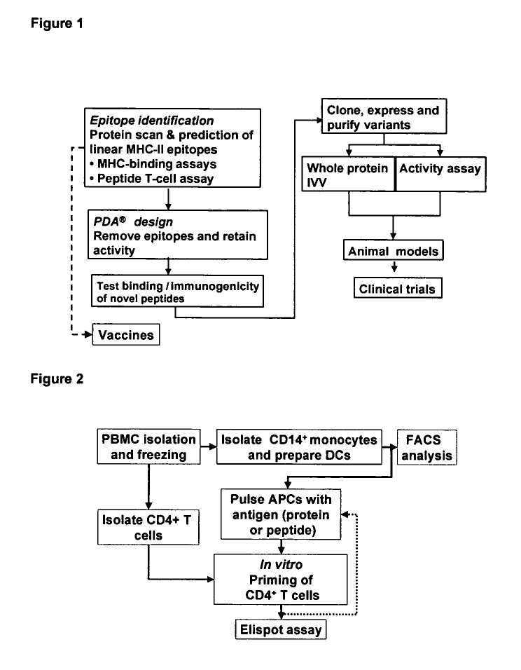

### DETAILED DESCRIPTION OF THE INVENTION

By “nine-mer peptide frame” and grammatical equivalents herein is meant a linear sequence of nine amino acids that is located in a protein of interest. nine-mer frames may be analyzed for their propensity to bind one or more class II MHC alleles. By “allele” and grammatical equivalents herein is meant an alternative form of a gene. Specifically, in the context of class II MHC molecules, alleles comprise all naturally occurring sequence variants of DRA, DRB1, DRB3/4/5, DQA-I, DQB1, DPA-I, and DPB1 molecules. By “Epo-responsive disorders” and grammatical equivalents herein is meant diseases, disorders, and conditions that can benefit from treatment with Epo. Examples of disorders that may benefit from treatment with EPO include, but are not limited to, cardiovascular diseases, particularly atherosclerosis, coronary artery disease, heart attack, stroke, and restenosis, diseases of lipid and cholesterol homeostasis, including conditions associated with EPO or HDL defects or deficiencies, analphalipoproteinemia, hypoalphalipoproteinemia, hyperlipidemia, amyloidosis, and viral and bacterial infections. By “hit” and grammatical equivalents herein is meant, in the context of the matrix method, that a given peptide is predicted to bind to a given class II MHC allele. In a preferred embodiment, a hit is defined to be a peptide with binding affinity among the top 5%, or 3%, or 1% of binding scores of random peptide sequences. In an alternate embodiment, a hit is defined to be a peptide with a binding affinity that exceeds some threshold, for instance a peptide that is predicted to bind an MHC allele with at least 100 μM or 10 μM or 1 μM affinity. By “immunogenicity” and grammatical equivalents herein is meant the ability of a protein to elicit an immune response, including but not limited to production of neutralizing and non-neutralizing antibodies, formation of immune complexes, complement activation, mast cell activation, inflammation, and anaphylaxis. By “reduced immunogenicity” and grammatical equivalents herein is meant a decreased ability to activate the immune system, when compared to the wild type protein. For example, a variant protein can be said to have “reduced immunogenicity” if it elicits neutralizing or non-neutralizing antibodies in lower titer or in fewer patients than the wild type protein. In a preferred embodiment, the probability of raising neutralizing antibodies is decreased by at least 5%, with at least 50% or 90% decreases being especially preferred. So, if a wild type produces an immune response in 10% of patients, a variant with reduced immunogenicity would produce an immune response in not more than 9.5% of patients, with less than 5% or less than 1% being especially preferred. A variant protein also can be said to have “reduced immunogenicity” if it shows decreased binding to one or more MHC alleles or if it induces T-cell activation in a decreased fraction of patients relative to the parent protein. In a preferred embodiment, the probability of T-cell activation is decreased by at least 5%, with at least 50% or 90% decreases being especially preferred. By “matrix method” and grammatical equivalents thereof herein is meant a method for calculating peptide—MHC affinity in which a matrix is used that contains a score for each possible residue at each position in the peptide, interacting with a given MHC allele. The binding score for a given peptide—MHC interaction is obtained by summing the matrix values for the amino acids observed at each position in the peptide. By “MHC-binding agretopes” and grammatical equivalents herein is meant peptides that are capable of binding to one or more class II MHC alleles with appropriate affinity to enable the formation of MHC-peptide-T-cell receptor complexes and subsequent T-cell activation. MHC-binding agretopes are linear peptide sequences that comprise at least approximately 9 residues. By “parent protein” as used herein is meant a protein that is subsequently modified to generate a variant protein. Said parent protein may be a wild-type or naturally occurring protein, or a variant or engineered version of a naturally occurring protein. “Parent protein” may refer to the protein itself, compositions that comprise the parent protein, or any amino acid sequence that encodes it. Accordingly, by “parent erythropoietn protein” as used herein is meant an erythropoietin protein that is modified to generate a variant erythropoietin protein. By “patient” herein is meant both humans and other animals, particularly mammals, and organisms. Thus the methods are applicable to both human therapy and veterinary applications. In the preferred embodiment the patient is a mammal, and in the most preferred embodiment the patient is human. By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids [see Simon et al., Proc. Natl. Acad. Sci. U.S.A. 89(20:9367-71 (1992), incorporated entirely by reference), generally depending on the method of synthesis. For example, homo-phenylalanine, citrulline, and noreleucine are considered amino acids for the purposes of the invention. “Amino acid” also includes amino acid residues such as proline and hydroxyproline. Both D- and L-amino acids may be utilized. By “treatment” herein is meant to include therapeutic treatment, as well as prophylactic, or suppressive measures for the disease or disorder. Thus, for example, successful administration of a variant EPO protein prior to onset of the disease may result in treatment of the disease. As another example, successful administration of a variant EPO protein after clinical manifestation of the disease to combat the symptoms of the disease comprises “treatment” of the disease. “Treatment” also encompasses administration of a variant EPO protein after the appearance of the disease in order to eradicate the disease. Successful administration of an agent after onset and after clinical symptoms have developed, with possible abatement of clinical symptoms and perhaps amelioration of the disease, further comprises “treatment” of the disease. By “variant erythropoietin nucleic acids” and grammatical equivalents herein is meant nucleic acids that encode variant erythropoietin proteins. Due to the degeneracy of the genetic code, an extremely large number of nucleic acids may be made, all of which encode the variant erythropoietin proteins of the present invention, by simply modifying the sequence of one or more codons in a way which does not change the amino acid sequence of the variant erythropoietin. By “variant erythropoietin proteins” and grammatical equivalents thereof herein is meant non-naturally occurring proteins which differ from the wild type or parent erythropoietin protein by at least 1 amino acid insertion, deletion, or substitution. Erythropoietin variants are characterized by the predetermined nature of the variation, a feature that sets them apart from naturally occurring allelic or interspecies variation of the erythropoietin protein sequence. The erythropoietin variants typically either exhibit biological activity that is comparable to naturally occurring erythropoietin or have been specifically engineered to have alternate biological properties. The variant erythropoietin proteins may contain insertions, deletions, and/or substitutions at the N-terminus, C-terminus, or internally. In a preferred embodiment, variant erythropoietin proteins have at least 1 residue that differs from the naturally occurring erythropoietin sequence, with at least 2, 3, 4, or 5 different residues being more preferred. Variant erythropoietin proteins may contain further modifications, for instance mutations that alter stability or solubility or which enable or prevent posttranslational modifications such as PEGylation or glycosylation. Variant erythropoietin proteins may be subjected to co- or post-translational modifications, including but not limited to synthetic derivatization of one or more side chains or termini, glycosylation, PEGylation, circular permutation, cyclization, fusion to proteins or protein domains, and addition of peptide tags or labels. By “wild type or wt” and grammatical equivalents thereof herein is meant an amino acid sequence or a nucleotide sequence that is found in nature and includes allelic variations; that is, an amino acid sequence or a nucleotide sequence that has not been intentionally modified. In a preferred embodiment, the wild type sequence is SEQ ID NO: 1.

Identification of MHC-binding Agretopes in Erythropoietin

MHC-binding peptides are obtained from proteins by a process called antigen processing. First, the protein is transported into an antigen presenting cell (APC) by endocytosis or phagocytosis. A variety of proteolytic enzymes then cleave the protein into a number of peptides. These peptides can then be loaded onto class II MHC molecules, and the resulting peptide-MHC complexes are transported to the cell surface. Relatively stable peptide-MHC complexes can be recognized by T-cell receptors that are present on the surface of naive T cells. This recognition event is required for the initiation of an immune response. Accordingly, blocking the formation of stable peptide-MHC complexes is an effective approach for preventing unwanted immune responses.

The factors that determine the affinity of peptide-MHC interactions have been characterized using biochemical and structural methods. Peptides bind in an extended conformation bind along a groove in the class II MHC molecule. While peptides that bind class II MHC molecules are typically approximately 13-18 residues long, a nine-residue region is responsible for most of the binding affinity and specificity. The peptide binding groove can be subdivided into “pockets”, commonly named P1 through P9, where each pocket is comprises the set of MHC residues that interacts with a specific residue in the peptide. A number of polymorphic residues face into the peptide-binding groove of the MHC molecule. The identity of the residues lining each of the peptide-binding pockets of each MHC molecule determines its peptide binding specificity. Conversely, the sequence of a peptide determines its affinity for each MHC allele.

Several methods of identifying MHC-binding agretopes in protein sequences are known in the art and may be used to identify agretopes in Epo. Sequence-based information can be used to determine a binding score for a given peptide - MHC interaction (see for example Mallios, Bioinformatics 15: 432-439 (1999); Mallios, Bioinformatics 17: p942-948 (2001); Stumiolo et al. Nature Biotech. 17: 555-561(1999), all incorporated entirely by reference). It is possible to use structure-based methods in which a given peptide is computationally placed in the peptide-binding groove of a given MHC molecule and the interaction energy is determined (for example, see WO 98/59244 and WO 02/069232, both incorporated entirely by reference). Such methods may be referred to as “threading” methods. Alternatively, purely experimental methods can be used; for example a set of overlapping peptides derived from the protein of interest can be experimentally tested for the ability to induce T-cell activation and/or other aspects of an immune response. (see for example WO 02/77187, incorporated entirely by reference).

In a preferred embodiment, MHC-binding propensity scores are calculated for each 9-residue frame along the erythropoietin sequence using a matrix method (see Stumiolo et al., supra; Marshall et al., J. Immunol. 154: 5927-5933 (1995), and Hammer et al., J. Exp. Med. 180: 2353-2358 (1994), both incorporated entirely by reference). It is also possible to consider scores for only a subset of these residues, or to consider also the identities of the peptide residues before and after the 9-residue frame of interest. The matrix comprises binding scores for specific amino acids interacting with the peptide binding pockets in different human class II MHC molecule. In the most preferred embodiment, the scores in the matrix are obtained from experimental peptide binding studies. In an alternate preferred embodiment, scores for a given amino acid binding to a given pocket are extrapolated from experimentally characterized alleles to additional alleles with identical or similar residues lining that pocket Matrices that are produced by extrapolation are referred to as “virtual matrices”.

In a preferred embodiment, the matrix method is used to calculate scores for each peptide of interest binding to each allele of interest. Several methods can then be used to determine whether a given peptide will bind with significant affinity to a given MHC allele. In one embodiment, the binding score for the peptide of interest is compared with the binding propensity scores of a large set of reference peptides. Peptides whose binding propensity scores are large compared to the reference peptides are likely to bind MHC and may be classified as “hits”. For example, if the binding propensity score is among the highest 1% of possible binding scores for that allele, it may be scored as a “hit” at the 1% threshold. The total number of hits at one or more threshold values is calculated for each peptide. In some cases, the binding score may directly correspond with a predicted binding affinity. Then, a hit may be defined as a peptide predicted to bind with at least 100 μM or 10 μM or 1 μM affinity.

In a preferred embodiment, the number of hits for each nine-mer frame in the protein is calculated using one or more threshold values ranging from 0.5% to 10%. In an especially preferred embodiment, the number of hits is calculated using 1%, 3%, and 5% thresholds.

In a preferred embodiment, MHC-binding agretopes are identified as the nine-mer frames that bind to several class II MHC alleles. In an especially preferred embodiment, MHC-binding agretopes are predicted to bind at least 10 alleles at 5% threshold and/or at least 5 alleles at 1% threshold. Such nine-mer frames may be especially likely to elicit an immune response in many members of the human population.

In a preferred embodiment, MHC-binding agretopes are predicted to bind MHC alleles that are present in at least 0.01-10% of the human population. Alternatively, to treat conditions that are linked to specific class II MHC alleles, MHC-binding agretopes are predicted to bind MHC alleles that are present in at least 0.01-10% of the relevant patient population.

Data about the prevalence of different MHC alleles in different ethnic and racial groups has been acquired by groups such as the National Marrow Donor Program (NMDP); for example see Mignot et al. Am. J. Hum. Genet 68: 686-699 (2001), Southwood et al. J. Immunol. 160: 3363-3373 (1998), Hurley et al. Bone Marrow Transplantation 25: 136-137 (2000), Sintasath Hum. Immunol. 60: 1001 (1999), Collins et al. Tissue Antigens 55: 48 (2000), Tang et al. Hum. Immunol. 63: 221 (2002), Chen et al. Hum. Immunol. 63: 665 (2002), Tang et al. Hum. Immunol. 61: 820 (2000), Gans et al. Tissue Antigens 59: 364-369, and Baldassarre et al. Tissue Antigens 61: 249-252 (2003), all incorporated entirely by reference.

In a preferred embodiment, MHC binding agretopes are predicted for MHC heterodimers comprising highly prevalent MHC alleles. Class II MHC alleles that are present in at least 10% of the US population include but are not limited to: DPA-I*0103, DPA-I*0201, DPB1*0201, DPB1*0401, DPB1*0402, DQA-I*0101, DQA-I*0102, DQA-I*0201, DQA-I*0501, DQB1*0201, DQB1*0202, DQB1*0301, DQB1*0302, DQB1*0501, DQB1*0602, DRA*0101, DRB1*0701, DRB1*1501, DRB1*0301, DRB1*0101, DRB1*1101, DRB1*1301, DRB3*0101, DRB3*0202, DRB4*0101, DRB4*0103, and DRB5*0101.

In a preferred embodiment, MHC binding agretopes are also predicted for MHC heterodimers comprising moderately prevalent MHC alleles. Class II MHC alleles that are present in 1% to 10% of the US population include but are not limited to: DPA-I*0104, DPA-I*0302, DPA-I*0301, DPB1*0101, DPB1*0202, DPB1*0301, DPB1*0501, DPB1*0601, DPB1*0901, DPB1*1001, DPB1*1101, DPB1*1301, DPB1*1401, DPB1*1501, DPB1*1701, DPB1*1901, DPB1*2001, DQA-I*0103, DQA-I*0104, DQA-I*0301, DQA-I*0302, DQA-I*0401, DQB1*0303, DQB1*0402, DQB1*0502, DQB1*0503, DQB1*0601, DQB1*0603, DRB1*1302, DRB1*0404, DRB1*0801, DRB1*0102, DRB1*1401, DRB1*1104, DRB1*1201, DRB1*1503, DRB1*0901, DRB1*1601, DRB1*0407, DRB1*1001, DRB1*1303, DRB1*0103, DRB1*1502, DRB1*0302, DRB1*0405, DRB1*0402, DRB1*1102, DRB1*0803, DRB1*0408, DRB1*1602, DRB1*0403, DRB3*0301, DRB5*0102, and DRB5*0202.

MHC binding agretopes may also be predicted for MHC heterodimers comprising less prevalent alleles. Information about MHC alleles in humans and other species can be obtained, for example, from the IMGT/HLA sequence database, part of the international ImMunoGeneTics project (IMGT).

In an especially preferred embodiment, an immunogenicity score is determined for each peptide, wherein said score depends on the fraction of the population with one or more MHC alleles that are hit at multiple thresholds. For example, the equation
Iscore=N(W₁P₁+W₃P₃+W₅P₅)
may be used, where P₁ is the percent of the population hit at 1%, P₃ is the percent of the population hit at 3%, P₅ is the percent of the population hit at 5%, each W is a weighting factor, and N is a normalization factor. In a preferred embodiment, W₁=10, W₃=5, W₅=2, and N is selected so that possible scores range from 0 to 100. In this embodiment, agretopes with Iscore greater than or equal to 10 are preferred and agretopes with Iscore greater than or equal to 25 are especially preferred.

In an additional preferred embodiment, MHC-binding agretopes are identified as the nine-mer frames that are located among “nested” agretopes, or overlapping 9-residue frames that are each predicted to bind a significant number of alleles. Such sequences may be especially likely to elicit an immune response.

Preferred MHC-binding agretopes are those agretopes that are predicted to bind, at a 3% threshold, to MHC alleles that are present in at least 5% of the population. Preferred MHC-binding agretopes in erythropoietin include, but are not limited to, agretope 1: residues 5-13; agretope 3: residues 51-59; agretope 5: residues 64-72; agretope 8: residues 75-83; agretope 10: residues 93-101; agretope 11: residues 102-110; agretope 13: residues 138-146; agretope 14: residues 141-149; agretope 15: residues 142-150; agretope 18: residues 149-157; agretope 19: residues 153-161.

Especially preferred MHC-binding agretopes are those agretopes that are predicted to bind, at a 1% threshold, to MHC alleles that are present in at least 10% of the population. Especially preferred MHC-binding agretopes in erythropoietin include, but are not limited to, agretope 3: residues 51-59; agretope 11: residues 102-110; agretope 13: residues 138-146; agretope 14: residues 141-149; agretope 15: residues 142-150; agretope 18: residues 149-157.

Alternate preferred MHC-binding agretopes are those agretopes that have Iscore greater than or equal to 10. Preferred MHC-binding agretopes in erythropoietin include, but are not limited to, agretope 1: residues 5-13; agretope 3: residues 51-59; agretope 5: residues 64-72; agretope 10: residues 93-101; agretope 11: residues 102-110; agretope 13: residues 138-146; agretope 14: residues 141-149; agretope 15: residues 142-150; agretope 18: residues 149-157; agretope 19: residues 153-161.

Alternate especially preferred MHC-binding agretopes are those agretopes that have Iscore greater than or equal to 25. Preferred MHC-binding agretopes in erythropoietin include, but are not limited to, agretope 3: residues 51-59; agretope 11: residues 102-110; agretope 13: residues 138-146; agretope 14: residues 141-149; agretope 15: residues 142-150; agretope 18: residues 149-157.

Additional especially preferred MHC-binding agretopes are those agretopes whose sequences partially overlap with additional MHC-binding agretopes. Sets of overlapping MHC-binding agretopes in erythropoietin include, but are not limited to, agretope 13: residues 138-146; agretope 14: residues 141-149; agretope 15: residues 142-150; agretope 18: residues 149-157.

Confirmation of MHC-binding Agretopes

In a preferred embodiment, the immunogenicity of the above-predicted MHC-binding agretopes is experimentally confirmed by measuring the extent to which peptides comprising each predicted agretope can elicit an immune response. However, it is possible to proceed from agretope prediction to agretope removal without the intermediate step of agretope confirmation.

Several methods, discussed in more detail below, can be used for experimental confirmation of agretopes. For example, sets of naive T cells and antigen presenting cells from matched donors can be stimulated with a peptide containing an agretope of interest, and T-cell activation can be monitored. It is also possible to first stimulate T cells with the whole protein of interest, and then re-stimulate with peptides derived from the whole protein. If sera are available from patients who have raised an immune response to Epo, it is possible to detect mature T cells that respond to specific epitopes. In a preferred embodiment, interferon gamma or IL-5 production by activated T-cells is monitored using Elispot assays, although it is also possible to use other indicators of T-cell activation or proliferation such as tritiated thymidine incorporation or production of other cytokines.

Patient Genotype Analysis and Screening

HLA genotype is a major determinant of susceptibility to specific autoimmune diseases (see for example Nepom Clin. Immunol. Immunopathol. 67: S50-S55 (1993)) and infections (see for example Singh et al. Emerg. Infect. Dis. 3: 41-49 (1997)), both incorporated entirely by reference. Furthermore, the set of MHC alleles present in an individual can affect the efficacy of some vaccines (see for example Cailat-Zucman et al. Kidney Int. 53: 1626-1630 (1998) and Poland et al. Vaccine 20: 430-438 (2001), both incorporated entirely by reference). HLA genotype may also confer susceptibility for an individual to elicit an unwanted immune response to an erythropoietin therapeutic.

In a preferred embodiment, class II MHC alleles that are associated with increased or decreased susceptibility to elicit an immune response to EPO proteins are identified. For example, patients treated with EPO therapeutics may be tested for the presence of anti-EPO antibodies and genotyped for class II MHC. Alternatively, T-cell activation assays such as those described above may be conducted using cells derived from a number of genotyped donors. Alleles that confer susceptibility to EPO immunogenicity may be defined as those alleles that are significantly more common in those who elicit an immune response versus those who do not. Similarly, alleles that confer resistance to EPO immunogenicity may be defined as those that are significantly less common in those who do not elicit an immune response versus those that do. It is also possible to use purely computational techniques to identify which alleles are likely to recognize EPO therapeutics.

In one embodiment, the genotype association data is used to identify patients who are especially likely or especially unlikely to raise an immune response to an EPO therapeutic.

Design of Active, Less-immunogenic Variants

In a preferred embodiment, the above-determined MHC-binding agretopes are replaced with alternate amino acid sequences to generate active variant EPO proteins with reduced or eliminated immunogenicity. Alternatively, the MHC-binding agretopes are modified to introduce one or more sites that are susceptible to cleavage during protein processing. If the agretope is cleaved before it binds to a MHC molecule, it will be unable to promote an immune response. There are several possible strategies for integrating methods for identifying less immunogenic sequences with methods for identifying structured and active sequences, including but not limited to those presented below.

In one embodiment, for one or more nine-mer agretope identified above, one or more possible alternate nine-mer sequences are analyzed for immunogenicity as well as structural and functional compatibility. The preferred alternate nine-mer sequences are then defined as those sequences that have low predicted immunogenicity and a high probability of being structured and active. It is possible to consider only the subset of nine-mer sequences that are most likely to comprise structured, active, less immunogenic variants. For example, it may be unnecessary to consider sequences that comprise highly non-conservative mutations or mutations that increase predicted immunogenicity.

In a preferred embodiment, less immunogenic variants of each agretope are predicted to bind MHC alleles in a smaller fraction of the population than the wild type agretope. In an especially preferred embodiment, the less immunogenic variant of each agretope is predicted to bind to MHC alleles that are present in not more than 5% of the population, with not more than 1% or 0.1% being most preferred.

Substitution Matrices

In another especially preferred embodiment, substitution matrices or other knowledge-based scoring methods are used to identify alternate sequences that are likely to retain the structure and function of the wild type protein. Such scoring methods can be used to quantify how conservative a given substitution or set of substitutions is. In most cases, conservative mutations do not significantly disrupt the structure and function of proteins (see for example, Bowie et al. Science 247: 1306-1310 (1990), Bowie and Sauer Proc. Nat. Acad. Sci. USA 86: 2152-2156 (1989), and Reidhaar-Olson and Sauer Proteins 7: 306-316 (1990), all incorporated entirely by reference). However, non-conservative mutations can destabilize protein structure and reduce activity (see for example, Lim et al. Biochem. 31: 4324-4333 (1992), incorporated entirely by reference). Substitution matrices including but not limited to BLOSUM62 provide a quantitative measure of the compatibility between a sequence and a target structure, which can be used to predict non-disruptive substitution mutations (see Topham et al. Prot. Eng. 10: 7-21 (1997), incorporated entirely by reference). The use of substitution matrices to design peptides with improved properties has been disclosed; see Adenot et al. J. Mol. Graph. Model. 17: 292-309 (1999), incorporated entirely by reference.

Substitution matrices include, but are not limited to, the BLOSUM matrices (Henikoff and Henikoff, Proc. Nat. Acad. Sci. USA 89: 10917 (1992), incorporated entirely by reference, the PAM matrices, the Dayhoff matrix, and the like. For a review of substitution matrices, see for example Henikoff Curr. Opin. Struct. Biol. 6: 353-360 (1996), incorporated entirely by reference. It is also possible to construct a substitution matrix based on an alignment of a given protein of interest and its homologs; see for example Henikoff and Henikoff Comput. Appl. Biosci. 12: 135-143 (1996), incorporated entirely by reference.

In a preferred embodiment, each of the substitution mutations that are considered has a BLOSUM62 score of zero or higher. According to this metric, preferred substitutions include, but are not limited to conservative mutations, shown in Table 1:

| Wild type | Preferred |
|---|---|
| residue | substitutions |
| A | CSTAGV |
| C | CA |
| D | SNDEQ |
| E | SNDEQHRK |
| F | MILFYW |
| G | SAGN |
| H | NEQHRY |
| I | MILVF |
| K | SNEQRK |
| L | MILVF |
| M | QMILVF |
| N | STGNDEQHRK |
| P | P |
| Q | SNDEQHRKM |
| R | NEQHRK |
| S | STAGNDEQK |
| T | TAMILV |
| V | STANV |
| W | FYW |
| Y | HFYW |

In addition, it is preferred that the total BLOSUM62 score of an alternate sequence for a nine residue MHC-binding agretope is decreased only modestly when compared to the BLOSUM62 score of the wild type nine residue agretope. In a preferred embodiment, the score of the variant nine-mer is at least 50% of the wild type score, with at least 67%, 75%, 80%, or 90% being especially preferred.

Alternatively, alternate sequences can be selected that minimize the absolute reduction in BLOSUM score; for example it is preferred that the score decrease for each nine-mer is less than 20, with score decreases of less than about 10 or about 5 being especially preferred. The exact value may be chosen to produce a library of alternate sequences that is experimentally tractable and also sufficiently diverse to encompass a number of active, stable, less immunogenic variants.

In a preferred embodiment, substitution mutations are preferentially introduced at positions that are substantially solvent exposed. As is known in the art, solvent exposed positions are typically more tolerant of mutation than positions that are located in the core of the protein.

In a preferred embodiment, substitution mutations are preferentially introduced at positions that are not highly conserved. As is known in the art, positions that are highly conserved among members of a protein family are often important for protein function, stability, or structure, while positions that are not highly conserved often may be modified without significantly impacting the structural or functional properties of the protein.

Alanine Substitutions

In an alternate embodiment, one or more alanine substitutions may be made, regardless of whether an alanine substitution is conservative or non-conservative. As is known in the art, incorporation of sufficient alanine substitutions may be used to disrupt intermolecular interactions.

In a preferred embodiment, variant nine-mers are selected such that residues that have been or can be identified as especially critical for maintaining the structure or function of erythropoietin retain their wild type identity. In alternate embodiments, it may be desirable to produce variant EPO proteins that do not retain wild type activity. In such cases, residues that have been identified as critical for function may be specifically targeted for modification.

Mutagenesis studies indicate four regions on EPO that are important for receptor binding and bioactivity: amino acids 11 to 15, 44 to 51, 100 to 108, and 147 to 151. These have been mapped to two sites: site 1 includes helix D (Asn147, Arg150, Gly15l and Leu155) and the A/B connecting loop (residues 42-51); site 2 includes helix A (Val11, Arg14, and Tyr15) and helix C (Ser100, Arg103, Ser104 and Leu108) (see Elliott et al. Blood 89: 493-502 (1997), Grodberg et al. Eur J Biochem 218: 597-601 (1993), Wen et al. J Biol Chem 269: 22839-22846 (1994), Kung/ et al. Arch Biochem Biophys 379: 85-89 (2000), all incorporated entirely by reference. C-terminal residues 154-159 have also been implicated (Bittorf et al. FEBS Left 336: 133-136 (1993), incorporated entirely by reference). Others have similarly identified two distinct receptor binding sites: site 1 (Arg150 and Lys152), which binds initially to one EpoR, and site 2 (Arg103, Ser104 and possibly Arg14) which binds to a second EpoR, and have postulated that both sites are required to form a homodimeric receptor complex required for EpoR activation. Arg103 appears to be particularly important for activity and may also play a stabilizing structural role (Matthews et al. Proc Natl Acad Sci USA 93: 9471-9476 (1996), Qiu et al. J Biol Chem 273: 11173-11176 (1998), both incorporated entirely by reference.

Removal of N-glycosylation sites (Asn24, Asn38, Asn83) has no effect on in vitro activity, but severely decreases in vivo activity, indicating that N-linked sugars are important for (1) proper biosynthesis and/or secretion and (2) expression of the in vivo activity probably by enhancing survival in the circulation. Aranesp®, the novel rHuEPO engineered to have two additional N-glycans, has a markedly prolonged half-life and therefore can be administered less frequently (see Yamaguchi et al. J Biol Chem 266: 20434-20966 (1991), Delorme et al. Biochemistry 31: 9871-9876 (1992), Bunn Blood 99: 1503 (2002), Jelkmann Eur J Haematol 69: 265-274 (2002), all incorporated entirely by reference.

Protein Design Methods

Protein design methods and MHC agretope identification methods may be used together to identify stable, active, and minimally immunogenic protein sequences (see WO03/006154, incorporated entirely by reference). The combination of approaches provides significant advantages over the prior art for immunogenicity reduction, as most of the reduced immunogenicity sequences identified using other techniques fail to retain sufficient activity and stability to serve as therapeutics.

Protein design methods may identify non-conservative or unexpected mutations that nonetheless confer desired functional properties and reduced immunogenicity, as well as identifying conservative mutations. Nonconservative mutations are defined herein to be all substitutions not included in Table 1 above; nonconservative mutations also include mutations that are unexpected in a given structural context, such as mutations to hydrophobic residues at the protein surface and mutations to polar residues in the protein core.

Furthermore, protein design methods may identify compensatory mutations. For example, if a given first mutation that is introduced to reduce immunogenicity also decreases stability or activity, protein design methods may be used to find one or more additional mutations that serve to recover stability and activity while retaining reduced immunogenicity. Similarly, protein design methods may identify sets of two or more mutations that together confer reduced immunogenicity and retained activity and stability, even in cases where one or more of the mutations, in isolation, fails to confer desired properties.

A wide variety of methods are known for generating and evaluating sequences. These include, but are not limited to, sequence profiling (Bowie and Eisenberg, Science 253(5016): 164-70, (1991)), residue pair potentials (Jones, Protein Science 3: 567-574, (1994)), and rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994)), all incorporated entirely by reference.

Protein Design Automation® (PDA®) Technology

In an especially preferred embodiment, rational design of improved erythropoietin variants is achieved by using Protein Design Automation® (PDA®) technology. (See U.S. Pat. Nos. 6,188,965; 6,269,312; 6,403,312; WO98/47089 and U.S. Ser. Nos. 09/058,459, 09/127,926, 60/104,612, 60/158,700, 09/419,351, 60/181,630, 60/186,904, 09/419,351, 09/782,004 and 09/927,790, 60/347,772, and 10/218,102; and PCT/US01/218,102 and U.S. Ser. No. 10/218,102, U.S. Ser. No. 60/345,805; U.S. Ser. No. 60/373,453 and U.S. Ser. No. 60/374,035, all incorporated entirely by reference.)

PDA® technology couples computational design algorithms that generate quality sequence diversity with experimental high-throughput screening to discover proteins with improved properties. The computational component uses atomic level scoring functions, side chain rotamer sampling, and advanced optimization methods to accurately capture the relationships between protein sequence, structure, and function. Calculations begin with the three-dimensional structure of the protein and a strategy to optimize one or more properties of the protein. PDA® technology then explores the sequence space comprising all pertinent amino acids (including unnatural amino acids, if desired) at the positions targeted for design. This is accomplished by sampling conformational states of allowed amino acids and scoring them using a parameterized and experimentally validated function that describes the physical and chemical forces governing protein structure. Powerful combinatorial search algorithms are then used to search through the initial sequence space, which may constitute 10⁵⁰ sequences or more, and quickly return a tractable number of sequences that are predicted to satisfy the design criteria. Useful modes of the technology span from combinatorial sequence design to prioritized selection of optimal single site substitutions. PDA® technology has been applied to numerous systems including important pharmaceutcal and industrial proteins and has a demonstrated record of success in protein optimization.

PDA® utilizes three-dimensional structural information. In a most preferred embodiment, the structure of EPO is determined using X-ray crystallography or NMR methods, which are well known in the art. The solution structure of free human EPO has been determined by NMR (Cheetham et al. Nat Struct Biol 5: 861-866 (1998), incorporated entirely by reference). The crystal structure of human EPO complexed to the extracellular ligand-binding domains of EpoR has been resolved at 1.9 Å and the crystal structure of the extracellular domain of EpoR in its unliganded form has been resolved at 2.4 Å. See Syed et al. Nature 395: 511-516 (1998), Livnah et al. Science 283: 987-990 (1999), incorporated entirely by reference.

In a preferred embodiment, the results of matrix method calculations are used to identify which of the 9 amino acid positions within the agretope(s) contribute most to the overall binding propensities for each particular allele “hit”. This analysis considers which positions (P1-P9) are occupied by amino acids which consistently make a significant contribution to MHC binding affinity for the alleles scoring above the threshold values. Matrix method calculations are then used to identify amino acid substitutions at said positions that would decrease or eliminate predicted immunogenicity and PDA® technology is used to determine which of the alternate sequences with reduced or eliminated immunogenicity are compatible with maintaining the structure and function of the protein.

In an alternate preferred embodiment, the residues in each agretope are first analyzed by one skilled in the art to identify alternate residues that are potentially compatible with maintaining the structure and function of the protein. Then, the set of resulting sequences are computationally screened to identify the least immunogenic variants. Finally, each of the less immunogenic sequences are analyzed more thoroughly in PDA® technology protein design calculations to identify protein sequences that maintain the protein structure and function and decrease immunogenicity.

In an alternate preferred embodiment, each residue that contributes significantly to the MHC binding affinity of an agretope is analyzed to identify a subset of amino acid substitutions that are potentially compatible with maintaining the structure and function of the protein. This step may be performed in several ways, including PDA® calculations or visual inspection by one skilled in the art. Sequences may be generated that contain all possible combinations of amino acids that were selected for consideration at each position. Matrix method calculations can be used to determine the immunogenicity of each sequence. The results can be analyzed to identify sequences that have significantly decreased immunogenicity. Additional PDA® calculations may be performed to determine which of the minimally immunogenic sequences are compatible with maintaining the structure and function of the protein.

In an alternate preferred embodiment, pseudo-energy terms derived from the peptide binding propensity matrices are incorporated directly into the PDAE technology calculations. In this way, it is possible to select sequences that are active and less immunogenic in a single computational step.

Combining Immunogenicity Reduction Strategies

In a preferred embodiment, more than one method is used to generate variant proteins with desired functional and immunological properties. For example, substitution matrices may be used in combination with PDA® technology calculations. Strategies for immunogenicity reduction include, but are not limited to, those described in U.S. Ser. No. 09/903,378; WO 01/21823; U.S. Ser. No. 10/039,170; WO 02/00165; U.S. Ser. Nos. 10/339,788; 10/754,296; and U.S. Ser. No. 10/638,995, all incorporated entirely by reference.

In a preferred embodiment, a variant protein with reduced binding affinity for one or more class II MHC alleles is further engineered to confer improved solubility. As protein aggregation may contribute to unwanted immune responses, increasing protein solubility may reduce immunogenicity. See U.S. Ser. No. 10/820,467, filed Mar. 30, 2004, entitled, “Interferon Variants With Improved Properties”, incorporated entirely by reference.

In an additional preferred embodiment, a variant protein with reduced binding affinity for one or more class II MHC alleles is further modified by derivitization with PEG or another molecule. As is known in the art, PEG may sterically interfere with antibody binding or improve protein solubility, thereby reducing immunogenicity. In an especially preferred embodiment, rational PEGylation methods are used. See, PCT/US2004/008425; and U.S. Ser. No. 10/ 10/956,352, filed Sep. 30, 2004, entitled, “Rational Chemical Modification,” both incorporated entirely by reference.

In a further preferred embodiment, a variant protein with reduced binding affinity for one or more class II MHC alleles is further modified by circular permutation or cyclization.

In a preferred embodiment, PDAS technology and matrix method calculations are used to remove more than one MHC-binding agretope from a protein of interest.

Generating the Variants

Variant interferon nucleic acids and proteins of the invention may be produced using a number of methods known in the art.

Preparing Nucleic Acids Encoding the EPO Variants

In a preferred embodiment, nucleic acids encoding EPO variants are prepared by total gene synthesis, or by site-directed mutagenesis of a nucleic acid encoding wild type or variant EPO protein. Methods including template-directed ligation, recursive PCR, cassette mutagenesis, site-directed mutagenesis or other techniques that are well known in the art may be utilized (see for example Strizhov et al. PNAS 93:15012-15017 (1996), Prodromou and Perl, Prot. Eng. 5: 827-829 (1992), Jayaraman and Puccini, Biotechniques 12: 392-398 (1992), and Chalmers et al. Biotechniques 30: 249-252 (2001), all incorporated entirely by reference).

In addition, it should be noted that variant EPO proteins and nucleic acids can be made that include substitutions in “fixed” or “non-agretope” positions as well. For example, while variants within an agretope are recited, any particular variant can include additional variants within non-agretope positions. In addition, any combination of recited variants can be made, as well as combinations of recited variants and non-agretope variants can be made.

Expression Vectors

In a preferred embodiment, an expression vector that comprises the components described below and a gene encoding a variant EPO protein is prepared. Numerous types of appropriate expression vectors and suitable regulatory sequences for a variety of host cells are known in the art. The expression vectors may contain transcriptional and translational regulatory sequences including but not limited to promoter sequences, ribosomal binding sites, transcriptional start and stop sequences, translational start and stop sequences, transcription terminator signals, polyadenylation signals, and enhancer or activator sequences. In a preferred embodiment, the regulatory sequences include a promoter and transcriptional start and stop sequences. In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences, which flank the expression construct. The integrating vector may be directed to a specific locus in the host cell by selecting the appropriate homologous sequence for inclusion in the vector. Constructs for integrating vectors are well known in the art. In addition, in a preferred embodiment, the expression vector contains a selectable marker gene to allow the selection of transformed host cells. Selection genes are well known in the art and will vary with the host cell used. The expression vectors may be either self-replicating extrachromosomal vectors or vectors which integrate into a host genome.

The expression vector may include a secretory leader sequence or signal peptide sequence that provides for secretion of the variant EPO protein from the host cell. Suitable secretory leader sequences that lead to the secretion of a protein are known in the art. The signal sequence typically encodes a signal peptide comprised of hydrophobic amino acids, which direct the secretion of the protein from the cell. The protein is either secreted into the growth media or, for prokaryotes, into the periplasmic space, located between the inner and outer membrane of the cell. For expression in bacteria, bacterial secretory leader sequences, operably linked to a variant EPO encoding nucleic acid, are usually preferred.

Transfection/Transformation

The variant EPO nucleic acids are introduced into the cells either alone or in combination with an expression vector in a manner suitable for subsequent expression of the nucleic acid. The method of introduction is largely dictated by the targeted cell type. Exemplary methods include CaPO₄ precipitation, liposome fusion, Lipofectin®, electroporation, viral infection, dextran-mediated transfection, polybrene mediated transfection, protoplast fusion, direct microinjection, etc. The variant EPO nucleic acids may stably integrate into the genome of the host cell or may exist either transiently or stably in the cytoplasm. As outlined herein, a particularly preferred method utilizes retroviral infection, as outlined in PCT/US97/01019, incorporated entirely by reference.

Hosts for the Expression of EPO Variants

Appropriate host cells for the expression of EPO variants include yeast, bacteria, archaebacteria, fungi, and insect and animal cells, including mammalian cells. Of particular interest are bacteria such as E. coli and Bacillus subtilis, fungi such as Saccharomyces cerevisiae, Pichia pastoris, and Neurospora, insects such as Drosophila melangaster and insect cell lines such as SF9, mammalian cell lines including 293, CHO, COS, Jurkat, NIH3T3, etc (see the ATCC cell line catalog, hereby expressly incorporated entirely by reference), as well as primary cell lines.

EPO variants can also be produced in more complex organisms, including but not limited to plants (such as corn, tobacco, and algae) and animals (such as chickens, goats, cows); see for example Dove, Nature Biotechnol. 20: 777-779 (2002), incorporated entirely by reference.

In one embodiment, the cells may be additionally genetically engineered, that is, contain exogenous nucleic acid other than the expression vector comprising the variant EPO nucleic acid.

Expression and Purification Methods

Variant EPO proteins of the invention and nucleic acids encoding them may be produced using a number of methods known in the art.

In a preferred embodiment, nucleic acids encoding the EPO variants are prepared by total gene synthesis, or by site-directed mutagenesis of a nucleic acid encoding a parent EPO protein. Methods including template-directed ligation, recursive PCR, cassette mutagenesis, site-directed mutagenesis or other techniques that are well known in the art may be utilized (see for example Strizhov et al. PNAS 93:15012-15017 (1996), Prodromou and Perl, Prot. Eng. 5: 827-829 (1992), Jayaraman and Puccini, Biotechniques 12: 392-398 (1992), and Chalmers et al. Biotechniques 30: 249-252 (2001), all incorporated entirely by reference).

In a preferred embodiment, EPO variants are cloned into an appropriate expression vector and expressed in E. coli (see McDonald, J. R., Ko, C., Mismer, D., Smith, D. J. and Collins, F. Biochim. Biophys. Acta 1090: 70-80 (1991), both incorporated entirely by reference). In an alternate preferred embodiment, EPO variants are expressed in mammalian cells, yeast, baculovirus, or in vitro expression systems. A number of expression systems and methods for their use are well known in the art (see Current Protocols in Molecular Biology, Wiley & Sons, and Molecular Cloning—A Laboratory Manual—3^{rd} Ed., Cold Spring Harbor Laboratory Press, New York (2001), incorporated entirely by reference). The choice of codons, suitable expression vectors and suitable host cells will vary depending on a number of factors, and may be easily optimized as needed.

In a preferred embodiment, the EPO variants are purified or isolated after expression. Standard purification methods include electrophoretic, molecular, immunological and chromatographic techniques, including ion exchange, hydrophobic, affinity, and reverse-phase HPLC chromatography, and chromatofocusing. For example, a EPO variant may be purified using a standard anti-recombinant protein antibody column. Ultrafiltration and diafiltration techniques, in conjunction with protein concentration, are also useful. For general guidance in suitable purification techniques, see Scopes, R., Protein Purification, Springer-Verlag, NY, 3rd ed. (1994), incorporated entirely by reference. The degree of purification necessary will vary depending on the desired use, and in some instances no purification will be necessary.

Protocols for the expression and purification of EPO have been disclosed for bacteria such as E coli, yeast, baculovirus, and mammalian expression systems, including CHO cells. See Lin et al. Proc Natl Acad Sci USA 82: 7580-7584 (1985), Han et al. Chin J Biotechnol 12: 227-233 (1996), Zhang et al. Zhongguo Yi Xue Ke Xue Yuan Xue Bao 19: 389-394 (1997), Lee-Huang Proc Natl Acad Sci USA 81: 2708-2712 (1984), Bill et al. Biochim Biophys Acta 1340: 13-20 (1997), Quelle et at Protein Expr Purif 3: 461469 (1992), and U.S. Pat. No. 4,703,008, all incorporated entirely by reference.

Assaying the Activity of the Variants

The variant EPO proteins of the invention may be tested for activity using any of a number of methods, including but not limited to those described below.

EpoR binding can be determined by measuring displacement of ¹²⁵I-rHuEPO in human erythroleukemia cells (Elliott et al. Blood 89: 493-502 (1997), incorporated entirely by reference.

In vitro bioactivity assays include cell proliferation (stimulation of thymidine uptake) in Epo-dependent or Epo-responsive cells including 32D cells with EpoR, primary murine erythroid spleen cells, murine erythroleukemia cells, and human leukemia cells, and Janus kinase 2 phosphorylation assays (Elliott et al. Blood 89: 493-502 (1997), Qiu et al. J Biol Chem 273: 11173-11176 (1998), Wen et al. J Biol Chem 269: 22839-22846 (1994), all incorporated entirely by reference. Epo's effects on numerous nonerythroid cells and tissues can also be measured (see Weiss Oncologist 8 Suppl 3: 18-29 (2003), incorporated entirely by reference). In vitro assays indicative of neuroprotective or anti-inflammatory effects include inhibition of TNF production by glial cells after neuronal death and measurement of the response of human PBMCs, rat glial cells or brain to lipopolysaccharide (LPS) (Villa et al. J Exp Med 198: 971-975 (2003), incorporated entirely by reference). In vivo assays include measurement of change in hematocrit in rodents and humans (Imai et al. Clin Exp Hypertens 17: 485-506 (1995), Sytkowski et al. Proc Natl Acad Sci USA 95: 1184-1188 (1998), Elliott et al. Nat Biotechnol 21: 414-421 (2003), all incorporated entirely by reference, and quality of life and survival in anemic cancer patients. A rat model of cerebral ischemia (middle cerebral artery occlusion) has been used: brains are histologically evaluated for reduction of inflammatory responses (astrocyte activation and recruitment of leukocytes and microglia) and inhibition of the release of inflammatory cytokines is determined; effects on LPS-induced TNF production can also be measured (Villa et al. J Exp Med 198: 971-975 (2003), incorporated entirely by reference). Preclinical models are also available for other CNS disorders including EAE, multiple sclerosis, spinal cord trauma, and light- or ischemia-induced retinal damage. A murine model can be used to measure Epo-induced angiogenesis in wound healing. See Weiss Oncologist 8 Suppl 3: 18-29 (2003), incorporated entirely by reference.

Determining the Immunogenicity of the Variants

In a preferred embodiment, the immunogenicity of the erythropoietin variants is determined experimentally to confirm that the variants do have reduced or eliminated immunogenicity relative to the parent protein.

In a preferred embodiment, ex vivo T-cell activation assays are used to experimentally quantitate immunogenicity. In this method, antigen presenting cells and naive T cells from matched donors are challenged with a peptide or whole protein of interest one or more times. Then, T cell activation can be detected using a number of methods, for example by monitoring production of cytokines or measuring uptake of tritiated thymidine. In the most preferred embodiment, interferon gamma production is monitored using Elispot assays (see Schmittel et al. J. Immunol. Meth., 24: 17-24 (2000), incorporated entirely by reference).

Other suitable T-cell assays include those disclosed in Meidenbauer, et al. Prostate 43, 88-100 (2000); Schultes, B. C and Whiteside, T. L., J. Immunol. Methods 279,1-15 (2003); and Stickler, et al., J. Immunotherapy, 23, 654-660 (2000), all incorporated entirely by reference.

In a preferred embodiment, the PBMC donors used for the above-described T-cell activation assays will comprise class II MHC alleles that are common in patients requiring treatment for erythropoietin responsive disorders. For example, for most diseases and disorders, it is desirable to test donors comprising all of the alleles that are prevalent in the population. However, for diseases or disorders that are linked with specific MHC alleles, it may be more appropriate to focus screening on alleles that confer susceptibility to erythropoietin responsive disorders.

In a preferred embodiment, the MHC haplotype of PBMC donors or patients that raise an immune response to the wild type or variant erythropoietin are compared with the MHC haplotype of patients who do not raise a response. This data may be used to guide preclinical and clinical studies as well as aiding in identification of patients who will be especially likely to respond favorably or unfavorably to the erythropoietin therapeutic.

In an alternate preferred embodiment, immunogenicity is measured in transgenic mouse systems. For example, mice expressing fully or partially human class II MHC molecules may be used.

In an alternate embodiment, immunogenicity is tested by administering the erythropoietin variants to one or more animals, including rodents and primates, and monitoring for antibody formation. Non-human primates with defined MHC haplotypes may be especially useful, as the sequences and hence peptide binding specificities of the MHC molecules in non-human primates may be very similar to the sequences and peptide binding specificities of humans. Similarly, genetically engineered mouse models expressing human MHC peptide-binding domains may be used (see for example Sonderstrup et al. Immunol. Rev. 172: 335-343 (1999) and Forsthuberetal. J. Immunol. 167: 119-125 (2001), both incorporated entirely by reference).

Formulation and Administration to Patients

Once made, the variant EPO proteins and nucleic acids of the invention find use in a number of applications. In a preferred embodiment, a variant EPO protein or nucleic acid is administered to a patient to treat an EPO related disorder.

The pharmaceutical compositions of the present invention comprise a variant EPO protein in a form suitable for administration to a patient. In a preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations.

Adjuvant materials suitable for use in compositions of the invention include compounds independently noted for erythropoietic stimulatory effects, such as testosterones, progenitor cell stimulators, insulin-like growth factor, prostaglandins, serotonin, cyclic AMP, prolactin and triiodothyzonine, as well as agents generally employed in treatment of aplastic anemia, such as methenolene, stanozolol and nandrolone [see, e.g., Resegotti, et al., Panminerva Medica, 23, 243-248 (1981); McGonigle, et al., Kidney Int., 25(2), 437-444 (1984); Pavlovic-Kantera, et al., Expt. Hematol., 8(Supp. 8), 283-291 (1980); and Kurtz, FEBS Letters, 14a(1), 105-108 (1982), all incorporated entirely by reference). Also contemplated as adjuvants are substances reported to enhance the effects of, or synergize, erythropoietin or asialo-EPO, such as the adrenergic agonists, thyroid hormones, androgens and BPA [see, Dunn, “Current Concepts in Erythropoiesis”, John Wiley and Sons (Chichester, England, 1983); Weiland, et al., Blut, 44(3), 173-175 (1982); Kalmanti, Kidney Int., 22, 383-391 (1982); Shahidi, New. Eng. J. Med., 289, 72-80 (1973); I Fisher, et al., Steroids, 30(6), 833-845 (1977); Urabe, et al., J. Exp. Med., 149, 1314-1325 (1979); and Billat, et al., Expt. Hematol., 10(1), 133-140 (1982), all incorporated entirely by reference) as well as the classes of compounds designated “hepatic erythropoietic factors” [see, Naughton, et al., Acta. Haemat., 69, 171-179 (1983), incorporated entirely by reference) and “erythrotropins” (as described by Congote, et al. in Abstract 364, Proceedings 7th International Congress of Endocrinology (Quebec City, Quebec, Jul. 1-7, 1984); Cingote, Biochem. Biophys. Res. Comm., 115(2), 447-483 (1983) and Congote, Anal. Biochem., 140, 428-433 (1984), all incorporated entirely by reference) and “erythrogenins” (as described in Rothman, et al., J. Surg. Oncol., 20, 105-108 (1982), incorporated entirely by reference). Preliminary screenings designed to measure erythropoietic responses of ex-hypoxic polycythemic mice pre-treated with either 5-.alpha.-dihydrotestosterone or nandrolone and then given erythlopoietin of the present invention have generated equivocal results.

Combinations of pharmaceutical compositions may be administered. Moreover, the compositions may be administered in combination with other therapeutics.

The administration of the variant EPO proteins of the present invention, preferably in the form of a sterile aqueous solution, may be done in a variety of ways, including, but not limited to, orally, subcutaneously, intravenously, intranasally, transdermally, intraperitoneally, intramuscularly, parenterally, intrapulmonary, vaginally, rectally, or intraocularly. In some instances, for example, the variant EPO protein may be directly applied as a solution or spray. Depending upon the manner of introduction, the pharmaceutical composition may be formulated in a variety of ways. In a preferred embodiment, a therapeutically effective dose of a variant EPO protein is administered to a patient in need of treatment. By “therapeutically effective dose” herein is meant a dose that produces the effects for which it is administered. The exact dose will depend on the purpose of the treatment, and will be ascertainable by one skilled in the art using known techniques. In a preferred embodiment, the concentration of the therapeutically active variant EPO protein in the formulation may vary from about 0.1 to about 100 weight %. In another preferred embodiment, the concentration of the variant EPO protein is in the range of 0.003 to 1.0 molar, with dosages from 0.03, 0.05, 0.1, 0.2, and 0.3 millimoles per kilogram of body weight being preferred. As is known in the art, adjustments for variant EPO protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

rHuEPO is often given 2-3 times/week; however, preferred routes of administration, dosing, etc. depend on the indication (see Weiss Oncologist 8 Suppl 3: 18-29 (2003), incorporated entirely by reference). Aranesp®, the novel rHuEPO engineered to have two additional N-glycans, has a markedly prolonged half-life and has the advantage that it can be administered less frequently (see Elliott et al. Nat Biotechnol 21: 414-421 (2003), incorporated entirely by reference).

In an alternate embodiment, variant EPO nucleic acids may be administered; i.e., “gene therapy” approaches may be used. In this embodiment, variant EPO nucleic acids are introduced into cells in a patient in order to achieve in vivo synthesis of a therapeutically effective amount of variant EPO protein. Variant EPO nucleic acids may be introduced using a number of techniques, including but not limited to transfection with liposomes, viral (typically retroviral) vectors, and viral coat protein-liposome mediated transfection (Dzau et al., Trends in Biotechnology 11:205-210 (1993), incorporated entirely by reference). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990), both incorporated entirely by reference. For review of gene marking and gene therapy protocols see Anderson et al., Science 256:808-813 (1992), incorporated entirely by reference.

While the foregoing invention has been described above, it will be clear to one skilled in the art that various changes and additional embodiments made be made without departing from the scope of the invention. All publications, patents, patent applications (provisional, utility and PCT) or other documents cited herein are incorporated entirely by references in their entirety.

### EXAMPLES

Example 1

### Identification of MHC-binding Agretopes in Erythropoietin

Matrix method calculations (Sturniolo, supra) were conducted using the parent erythropoietin sequence shown in SEQ ID NO: 1.

Agretopes were predicted for the following alleles, each of which is present in at least 1% of the US population: DRB1*0101, DRB1*0102, DRB1*0301, DRB1*0401, DRB1*0402, DRB1*0404, DRB1*0405, DRB1*0408, DRB1*0701, DRB1*0801, DRB1*1101, DRB1*1102, DRB1*1104, DRB1*1301, DRB1*1302, DRB1*1501, and DRB1*1502.

For each nine-mer that is predicted to bind to at least one allele at a 5% threshold, the number of alleles that are hit at 1%, 3%, and 5% thresholds were given, as well as the percent of the US population that are predicted to react to the nine-mer. The worst nine-mers are shown in bold. They are predicted to be immunogenic in at least 10% of the US population, using a 1% threshold.

TABLE 2

Predicted MHC-binding agretopes in erythro-
poietin. The immunogenicity score, number of
alleles, and percent of population hit at 1%,
3%, and 5% thresholds are shown. Especially
preferred agretopes are predicted to affect at
least 10% of the population, using a 1%
threshold. (Residues from SEQ ID NO: 1)

| | | | | | | | | | |
|---|---|---|---|---|---|---|---|---|---|
| A- | | | | | | | | | |
| gre- | | | | | | | | | |
| tope | Res- | | | | | | | | |
| num- | i- | | | 1% | 3% | 5% | 1% | 3% | 5% |
| ber | dues | Sequence | IScore | hits | hits | hits | pop | pop | pop |

| | | | | | | | | | |
|---|---|---|---|---|---|---|---|---|---|
| A- | 5- | LICDSRVLE | 13.08 | 0 | 1 | 1 | 0% | 21% | 21% |
| gre- | 13 | | | | | | | | |
| tope | | | | | | | | | |
| 1 | | | | | | | | | |
| A- | 48- | FYAWKRMEV | 5.87 | 0 | 1 | 3 | 0% | 2% | 21% |
| gre- | 56 | | | | | | | | |
| tope | | | | | | | | | |
| 2 | | | | | | | | | |
| A- | 51- | WKRMEVGQQ | 34.02 | 4 | 7 | 7 | 18% | 37% | 37% |
| gre- | 59 | | | | | | | | |
| tope | | | | | | | | | |
| 3 | | | | | | | | | |
| A- | 61- | VEVWQGLAL | 5.55 | 0 | 0 | 1 | 0% | 0% | 23% |
| gre- | 69 | | | | | | | | |
| tope | | | | | | | | | |
| 4 | | | | | | | | | |
| A- | 64- | WQGLALLSE | 10.15 | 0 | 2 | 3 | 0% | 16% | 17% |
| gre- | 72 | | | | | | | | |
| tope | | | | | | | | | |
| 5 | | | | | | | | | |
| A- | 69- | LLSEAVLRG | 3.44 | 0 | 0 | 1 | 0% | 0% | 14% |
| gre- | 77 | | | | | | | | |
| tope | | | | | | | | | |
| 6 | | | | | | | | | |
| A- | 74- | VLRGQALLV | 6.50 | 0 | 0 | 2 | 0% | 0% | 27% |
| gre- | 82 | | | | | | | | |
| tope | | | | | | | | | |
| 7 | | | | | | | | | |
| A- | 75- | LRGQALLVK | 8.54 | 0 | 2 | 4 | 0% | 6% | 26% |
| gre- | 83 | | | | | | | | |
| tope | | | | | | | | | |
| 8 | | | | | | | | | |
| A- | 80- | LLVKSSQPW | 0.42 | 0 | 0 | 1 | 0% | 0% | 2% |
| gre- | 88 | | | | | | | | |
| tope | | | | | | | | | |
| 9 | | | | | | | | | |
| A- | 93- | LHVDKAVSG | 13.08 | 0 | 1 | 1 | 0% | 21% | 21% |
| gre- | 101 | | | | | | | | |
| tope | | | | | | | | | |
| 10 | | | | | | | | | |
| A- | 102- | LRSLTTLLR | 63.03 | 6 | 11 | 13 | 40% | 59% | 70% |
| gre- | 110 | | | | | | | | |
| tope | | | | | | | | | |
| 11 | | | | | | | | | |
| A- | 109- | LRALGAQKE | 2.31 | 0 | 1 | 2 | 0% | 2% | 7% |
| gre- | 117 | | | | | | | | |
| tope | | | | | | | | | |
| 12 | | | | | | | | | |
| A- | 138- | FRKLFRVYS | 42.19 | 2 | 8 | 9 | 19% | 45% | 57% |
| gre- | 146 | | | | | | | | |
| tope | | | | | | | | | |
| 13 | | | | | | | | | |
| A- | 141- | LFRVYSNFL | 32.60 | 1 | 1 | 4 | 25% | 25% | 34% |
| gre- | 149 | | | | | | | | |
| tope | | | | | | | | | |
| 14 | | | | | | | | | |
| A- | 142- | FRVYSNFLR | 34.32 | 2 | 4 | 4 | 24% | 32% | 32% |
| gre- | 150 | | | | | | | | |
| tope | | | | | | | | | |
| 15 | | | | | | | | | |
| A- | 144- | VYSNFLRGK | 0.42 | 0 | 0 | 1 | 0% | 0% | 2% |
| gre- | 152 | | | | | | | | |
| tope | | | | | | | | | |
| 16 | | | | | | | | | |
| A- | 145- | YSNFLRGKL | 1.23 | 0 | 1 | 1 | 0% | 2% | 2% |
| gre- | 153 | | | | | | | | |
| tope | | | | | | | | | |
| 17 | | | | | | | | | |
| A- | 149- | LRGKLKLYT | 32.03 | 3 | 6 | 6 | 17% | 36% | 36% |
| gre- | 157 | | | | | | | | |
| tope | | | | | | | | | |
| 18 | | | | | | | | | |
| A- | 153- | LKLYTGEAC | 14.26 | 0 | 1 | 2 | 0% | 23% | 24% |
| gre- | 161 | | | | | | | | |
| tope | | | | | | | | | |
| 19 | | | | | | | | | |

Alleles that are predicted as “hits” for each of the agretopes above are shown in the table below. “1” indicates a hit using a 1% threshold, “3” indicates a hit using a 3% threshold, and “5” indicates a hit using a 5% threshold.

TABLE 3
Predicted MHC-binding agretopes in erythropoietin. DRB1 alleles that are
predicted to bind to each allele at 1%, 3%, and 5% cutoffs are marked with “1”, “3”, or
“5”, respectively.

| Agretope | | | | | | | | | | | | | | | | | |
|---|---|---|---|---|---|---|---|---|---|---|---|---|---|---|---|---|---|
| number | 0101 | 0102 | 0301 | 0401 | 0402 | 0404 | 0405 | 0408 | 0701 | 0801 | 1101 | 1102 | 1104 | 1301 | 1302 | 1501 | 1502 |
| Agretope 1 | — | — | 3 | — | — | — | — | — | — | — | — | — | — | — | — | — | — |
| Agretope 2 | — | — | — | — | — | — | — | — | — | — | 5 | — | — | — | 5 | 10 | 3 |
| Agretope 3 | — | — | — | 3 | — | 3 | 1 | 1 | — | 1 | 1 | 10 | 3 | — | 10 | — | — |
| Agretope 4 | — | — | — | — | — | — | — | — | — | — | — | — | — | — | — | 5 | 10 |
| Agretope 5 | — | — | — | — | — | — | 5 | — | — | 3 | 3 | — | — | — | 10 | — | — |
| Agretope 6 | — | — | 5 | — | — | — | — | — | — | — | — | — | — | — | — | — | — |
| Agretope 7 | 10 | 5 | — | — | — | — | — | — | 10 | — | — | — | — | — | — | 5 | 10 |
| Agretope 8 | — | — | — | 10 | — | — | — | — | — | — | 5 | 3 | 3 | 5 | 10 | — | — |
| Agretope 9 | — | — | — | — | 5 | — | — | — | — | — | — | — | — | — | — | — | — |
| Agretope 10 | — | — | 3 | — | — | — | — | — | — | — | — | — | — | — | — | — | — |
| Agretope 11 | 10 | 5 | 3 | 1 | 3 | 1 | 3 | 1 | — | — | 1 | 3 | 1 | 1 | 3 | 5 | 10 |
| Agretope 12 | 10 | 5 | — | — | — | — | 3 | — | — | 10 | — | — | — | — | — | — | — |
| Agretope 13 | 3 | 10 | 5 | — | — | — | — | 10 | — | 3 | 1 | 3 | 3 | 3 | 1 | — | 3 |
| Agretope 14 | 10 | 5 | — | — | — | 5 | 5 | 10 | 1 | — | — | — | — | — | — | 10 | 10 |
| Agretope 15 | — | — | — | — | 10 | 10 | 10 | 3 | — | 10 | — | — | — | 10 | 3 | 1 | 1 |
| Agretope 16 | — | — | — | — | — | — | — | — | — | — | — | 5 | — | 10 | — | — | — |
| Agretope 17 | — | — | — | — | — | — | — | — | — | — | — | — | — | — | — | 10 | 3 |
| Agretope 18 | — | — | — | — | — | — | — | — | — | 1 | 3 | 1 | 3 | 1 | 3 | 10 | — |
| Agretope 19 | — | — | — | — | — | — | — | — | — | — | — | — | — | — | — | 3 | 5 |

### Example 2

### Identification of Suitable Less Immunogenic Sequences for MHC-binding Agretopes in Erythropoietin as Determined by BLOSUM Method

MHC-binding agretopes that were predicted to bind alleles present in at least 10% of the US population, using a 1% threshold, were analyzed to identify suitable less immunogenic variants.

At each agretope, all possible combinations of amino acid substitutions were considered, with the following requirements: (1) each substitution has a score of 0 or greater in the BLOSUM62 substitution matrix, (2) each substitution is capable of conferring reduced binding to at least one of the MHC alleles considered, and (3) once sufficient substitutions are incorporated to prevent any allele hits at a 1% threshold, no additional substitutions are added to that sequence.

Alternate sequences were scored for immunogenicity and structural compatibility. Preferred alternate sequences were defined to be those sequences that are not predicted to bind to any of the 17 MHC alleles tested above using a 1% threshold, and that have a total BLOSUM62 score that is at least 85% of the wild type score. In Tables 4-9, B(wt) is the BLOSUM62 score of the wild type nine-mer, I(alt) is the percent of the US population containing one or more MHC alleles that are predicted to bind the alternate nine-mer at a 1% threshold, and B(alt) is the BLOSUM62 score of the alternate nine-mer.

TABLE 4

Suitable less immunogenic variants of agretope
3 (residues 51-59).

| | | | | WT se- | |
|---|---|---|---|---|---|
| | | | | quence of | |
| | Variant | I | B | SEQ ID | B |
| Sequence ID | sequence | (alt) | (alt) | NO: 1 | (wt) |
| SEQ ID NO: 2 | WSRMEVGQQ | 0 | 46 | WKRMEVGQQ | 51 |
| SEQ ID NO: 3 | WKEMEVGQQ | 0 | 46 | WKRMEVGQQ | 51 |
| SEQ ID NO: 4 | WKRQEVGQQ | 0 | 47 | WKRMEVGQQ | 51 |
| SEQ ID NO: 5 | WKRMEMGQQ | 0 | 48 | WKRMEVGQQ | 51 |
| SEQ ID NO: 6 | WKRMEVGQR | 0 | 47 | WKRMEVGQQ | 51 |
| SEQ ID NO: 7 | WKRNEVGQK | 0 | 47 | WKRMEVGQQ | 51 |
| SEQ ID NO: 8 | WNRVEVGQQ | 0 | 42 | WKRMEVGQQ | 51 |
| SEQ ID NO: 9 | WNRMEAGQQ | 0 | 43 | WKRMEVGQQ | 51 |
| SEQ ID NO: 10 | WNRMELGQQ | 0 | 43 | WKRMEVGQQ | 51 |
| SEQ ID NO: 11 | WEQMEVGQQ | 0 | 43 | WKRMEVGQQ | 51 |
| SEQ ID NO: 12 | WEHMEVGQQ | 0 | 42 | WKRMEVGQQ | 51 |
| SEQ ID NO: 13 | WEKMEVGQQ | 0 | 44 | WKRMEVGQQ | 51 |
| SEQ ID NO: 14 | WERIEVGQQ | 0 | 43 | WKRMEVGQQ | 51 |
| SEQ ID NO: 15 | WERLEVGQQ | 0 | 44 | WKRMEVGQQ | 51 |
| SEQ ID NO: 16 | WERVEVGQQ | 0 | 43 | WKRMEVGQQ | 51 |
| SEQ ID NO: 17 | WERFEVGQQ | 0 | 43 | WKRMEVGQQ | 51 |
| SEQ ID NO: 18 | WERMEAGQQ | 0 | 44 | WKRMEVGQQ | 51 |
| SEQ ID NO: 19 | WERMEIGQQ | 0 | 46 | WKRMEVGQQ | 51 |
| SEQ ID NO: 20 | WERMELGQQ | 0 | 44 | WKRMEVGQQ | 51 |
| SEQ ID NO: 21 | WERMEVGQS | 0 | 42 | WKRMEVGQQ | 51 |
| SEQ ID NO: 22 | WERMEVGQN | 0 | 42 | WKRMEVGQQ | 51 |
| SEQ ID NO: 23 | WERMEVGQD | 0 | 42 | WKRMEVGQQ | 51 |
| SEQ ID NO: 24 | WERMEVGQM | 0 | 42 | WKRMEVGQQ | 51 |
| SEQ ID NO: 25 | WKNVEVGQQ | 0 | 42 | WKRMEVGQQ | 51 |
| SEQ ID NO: 26 | WKNMEAGQQ | 0 | 43 | WKRMEVGQQ | 51 |
| SEQ ID NO: 27 | WKQIEVGQQ | 0 | 43 | WKRMEVGQQ | 51 |
| SEQ ID NO: 28 | WKQVEVGQQ | 0 | 43 | WKRMEVGQQ | 51 |
| SEQ ID NO: 29 | WKQFEVGQQ | 0 | 43 | WKRMEVGQQ | 51 |
| SEQ ID NO: 30 | WKQMEAGQQ | 0 | 44 | WKRMEVGQQ | 51 |
| SEQ ID NO: 31 | WKQMELGQQ | 0 | 44 | WKRMEVGQQ | 51 |
| SEQ ID NO: 32 | WKQMEVGQN | 0 | 42 | WKRMEVGQQ | 51 |
| SEQ ID NO: 33 | WKQMEVGQM | 0 | 42 | WKRMEVGQQ | 51 |
| SEQ ID NO: 34 | WKHVEVGQQ | 0 | 42 | WKRMEVGQQ | 51 |
| SEQ ID NO: 35 | WKHFEVGQQ | 0 | 42 | WKRMEVGQQ | 51 |
| SEQ ID NO: 36 | WKHMEAGQQ | 0 | 43 | WKRNEVGQQ | 51 |
| SEQ ID NO: 37 | WKHMELGQQ | 0 | 43 | WKRNEVGQQ | 51 |
| SEQ ID NO: 38 | WKKIEVGQQ | 0 | 44 | WKRMEVGQQ | 51 |
| SEQ ID NO: 39 | WKKVEVGQQ | 0 | 44 | WKRMEVGQQ | 51 |
| SEQ ID NO: 40 | WKKFEVGQQ | 0 | 44 | WKRMEVGQQ | 51 |
| SEQ ID NO: 41 | WKKMEAGQQ | 0 | 45 | WKRMEVGQQ | 51 |
| SEQ ID NO: 42 | WKKMELGQQ | 0 | 45 | WKRMEVGQQ | 51 |
| SEQ ID NO: 43 | WKKMEVGQN | 0 | 43 | WKRMEVGQQ | 51 |
| SEQ ID NO: 44 | WKKMEVGQM | 0 | 43 | WKRMEVGQQ | 51 |
| SEQ ID NO: 45 | WKRIEAGQQ | 0 | 44 | WKRMEVGQQ | 51 |
| SEQ ID NO: 46 | WKRIELGQQ | 0 | 44 | WKRMEVGQQ | 51 |
| SEQ ID NO: 47 | WKRIEVGQN | 0 | 42 | WKRMEVGQQ | 51 |
| SEQ ID NO: 48 | WKRIEVGQD | 0 | 42 | WKRMEVGQQ | 51 |
| SEQ ID NO: 49 | WKRIEVGQM | 0 | 42 | WKRMEVGQQ | 51 |
| SEQ ID NO: 50 | WKRLEAGQQ | 0 | 45 | WKRMEVGQQ | 51 |
| SEQ ID NO: 51 | WKRLELGQQ | 0 | 45 | WKRMEVGQQ | 51 |
| SEQ ID NO: 52 | WKRLEVGQN | 0 | 43 | WKRMEVGQQ | 51 |
| SEQ ID NO: 53 | WKRLEVGQM | 0 | 43 | WKRMEVGQQ | 51 |
| SEQ ID NO: 54 | WKRVEAGQQ | 0 | 44 | WKRMEVGQQ | 51 |
| SEQ ID NO: 55 | WKRVEIGQQ | 0 | 46 | WKRMEVGQQ | 51 |
| SEQ ID NO: 56 | WKRVELGQQ | 0 | 44 | WKRMEVGQQ | 51 |
| SEQ ID NO: 57 | WKRVEVGQN | 0 | 42 | WKRMEVGQQ | 51 |
| SEQ ID NO: 58 | WKRVEVGQD | 0 | 42 | WKRMEVGQQ | 51 |
| SEQ ID NO: 59 | WKRVEVGQM | 0 | 42 | WKRMEVGQQ | 51 |
| SEQ ID NO: 60 | WKRFEAGQQ | 0 | 44 | WKRMEVGQQ | 51 |
| SEQ ID NO: 61 | WKRFEIGQQ | 0 | 46 | WKRMEVGQQ | 51 |
| SEQ ID NO: 62 | WKRFELGQQ | 0 | 44 | WKRMEVGQQ | 51 |
| SEQ ID NO: 63 | WKRFEVGQN | 0 | 42 | WKRMEVGQQ | 51 |
| SEQ ID NO: 64 | WKRFEVGQM | 0 | 42 | WKRMEVGQQ | 51 |
| SEQ ID NO: 65 | WKRMEAGQS | 0 | 43 | WKRMEVGQQ | 51 |
| SEQ ID NO: 66 | WKRMEAGQN | 0 | 43 | WKRMEVGQQ | 51 |
| SEQ ID NO: 67 | WKRMEAGQD | 0 | 43 | WKRMEVGQQ | 51 |
| SEQ ID NO: 68 | WKRMEAGQM | 0 | 43 | WKRMEVGQQ | 51 |
| SEQ ID NO: 69 | WKRMELGQS | 0 | 43 | WKRMEVGQQ | 51 |
| SEQ ID NO: 70 | WKRMELGQN | 0 | 43 | WKRMEVGQQ | 51 |
| SEQ ID NO: 71 | WKRMELGQD | 0 | 43 | WKRMEVGQQ | 51 |
| SEQ ID NO: 72 | WKRMELGQM | 0 | 43 | WKRMEVGQQ | 51 |
| SEQ ID NO: 73 | WNQMEIGQQ | 0 | 41 | WKRMEVGQQ | 51 |
| SEQ ID NO: 74 | WNKMEIGQQ | 0 | 42 | WKRMEVGQQ | 51 |
| SEQ ID NO: 75 | WNRIEIGQQ | 0 | 41 | WKRMEVGQQ | 51 |
| SEQ ID NO: 76 | WKNIEIGQQ | 0 | 41 | WKRMEVGQQ | 51 |
| SEQ ID NO: 77 | WKQLEIGQQ | 0 | 43 | WKRMEVGQQ | 51 |
| SEQ ID NO: 78 | WKQLEVGQE | 0 | 41 | WKRMEVGQQ | 51 |
| SEQ ID NO: 79 | WKQMEIGQS | 0 | 41 | WKRMEVGQQ | 51 |
| SEQ ID NO: 80 | WKQMEIGQD | 0 | 41 | WKRMEVGQQ | 51 |
| SEQ ID NO: 81 | WKHIEIGQQ | 0 | 41 | WKRMEVGQQ | 51 |
| SEQ ID NO: 82 | WKHLEIGQQ | 0 | 42 | WKRMEVGQQ | 51 |
| SEQ ID NO: 83 | WKKLEIGQQ | 0 | 44 | WKRMEVGQQ | 51 |
| SEQ ID NO: 84 | WKKLEVGQE | 0 | 42 | WKRMEVGQQ | 51 |
| SEQ ID NO: 85 | WKKMEIGQS | 0 | 42 | WKRMEVGQQ | 51 |
| SEQ ID NO: 86 | WKKMEIGQD | 0 | 42 | WKRMEVGQQ | 51 |
| SEQ ID NO: 87 | WKRIEIGQE | 0 | 43 | WKRMEVGQQ | 51 |
| SEQ ID NO: 88 | WKRIEIGQH | 0 | 41 | WKRMEVGQQ | 51 |
| SEQ ID NO: 89 | WKRLEIGQD | 0 | 42 | WKRMEVGQQ | 51 |

TABLE 5

Suitable less immunogenic variants of agretope
11 (residues 102-110).

| | | | | WT se- | |
|---|---|---|---|---|---|
| | | | | quence of | |
| | Variant | I | B | SEQ ID | B |
| Sequence ID | sequence | (alt) | (alt) | NO: 1 | (wt) |
| SEQ ID NO: 90 | LNSLTTLLR | 0 | 35 | LRSLTTLLR | 40 |
| SEQ ID NO: 91 | LESLTTLLR | 0 | 35 | LRSLTTLLR | 40 |
| SEQ ID NO: 92 | LQSLTTLLR | 0 | 36 | LRSLTTLLR | 40 |
| SEQ ID NO: 93 | LHSLTTLLR | 0 | 35 | LRSLTTLLR | 40 |
| SEQ ID NO: 94 | LKSLTTLLR | 0 | 37 | LRSLTTLLR | 40 |
| SEQ ID NO: 95 | LRDLTTLLR | 0 | 36 | LRSLTTLLR | 40 |
| SEQ ID NO: 96 | LRELTTLLR | 0 | 36 | LRSLTTLLR | 40 |
| SEQ ID NO: 97 | LRTLTSLLR | 0 | 33 | LRSLTTLLR | 40 |
| SEQ ID NO: 98 | LRTLTALLR | 0 | 32 | LRSLTTLLR | 40 |
| SEQ ID NO: 99 | LRALTSLLR | 0 | 33 | LRSLTTLLR | 40 |
| SEQ ID NO: | LRALTALLR | 0 | 32 | LRSLTTLLR | 40 |
| 100 | | | | | |
| SEQ ID NO: | LRQLTSLLR | 0 | 32 | LRSLTTLLR | 40 |
| 101 | | | | | |
| SEQ ID NO: | LRKLTSLLR | 0 | 32 | LRSLTTLLR | 40 |
| 102 | | | | | |
| SEQ ID NO: | LRSITALLR | 0 | 33 | LRSLTTLLR | 40 |
| 103 | | | | | |
| SEQ ID NO: | LRSVTSLLR | 0 | 33 | LRSLTTLLR | 40 |
| 104 | | | | | |
| SEQ ID NO: | LRSVTALLR | 0 | 32 | LRSLTTLLR | 40 |
| 105 | | | | | |
| SEQ ID NO: | LRSVTNLLR | 0 | 32 | LRSLTTLLR | 40 |
| 106 | | | | | |
| SEQ ID NO: | LRSVTTVLR | 0 | 34 | LRSLTTLLR | 40 |
| 107 | | | | | |
| SEQ ID NO: | LRSVTTFLR | 0 | 33 | LRSLTTLLR | 40 |
| 108 | | | | | |
| SEQ ID NO: | LRSLTSILR | 0 | 34 | LRSLTTLLR | 40 |
| 109 | | | | | |
| SEQ ID NO: | LRSLTSVLR | 0 | 33 | LRSLTTLLR | 40 |
| 110 | | | | | |
| SEQ ID NO: | LRSLTSFLR | 0 | 32 | LRSLTTLLR | 40 |
| 111 | | | | | |
| SEQ ID NO: | LRSLTAMLR | 0 | 33 | LRSLTTLLR | 40 |
| 112 | | | | | |
| SEQ ID NO: | LRSLTNILR | 0 | 33 | LRSLTTLLR | 40 |
| 113 | | | | | |
| SEQ ID NO: | LRSLTNVLR | 0 | 32 | LRSLTTLLR | 40 |
| 114 | | | | | |
| SEQ ID NO: | LRTVTTILR | 0 | 32 | LRSLTTLLR | 40 |
| 115 | | | | | |
| SEQ ID NO: | LRAVTTILR | 0 | 32 | LRSLTTLLR | 40 |
| 116 | | | | | |

TABLE 6

Suitable less immunogenic variants of agretope
13 (residues 138-146).

| | | | | WT sequence | |
|---|---|---|---|---|---|
| | Variant | I | B | of SEQ ID | B |
| Sequence ID | sequence | (alt) | (alt) | NO: 1 | (wt) |
| SEQ ID NO: | FNKLFRVYS | 0 | 41 | FRKLFRVYS | 46 |
| 117 | | | | | |
| SEQ ID NO: | FEKLFRVYS | 0 | 41 | FRKLFRVYS | 46 |
| 118 | | | | | |
| SEQ ID NO: | FQKLFRVYS | 0 | 42 | FRKLFRVYS | 46 |
| 119 | | | | | |
| SEQ ID NO: | FHKLFRVYS | 0 | 41 | FRKLFRVYS | 46 |
| 120 | | | | | |
| SEQ ID NO: | FKKLFRVYS | 0 | 43 | FRKLFRVYS | 46 |
| 121 | | | | | |
| SEQ ID NO: | FRELFRVYS | 0 | 42 | FRKLFRVYS | 46 |
| 122 | | | | | |
| SEQ ID NO: | FRKLFEVYS | 0 | 41 | FRKLFRVYS | 46 |
| 123 | | | | | |
| SEQ ID NO: | FRKLFQVYS | 0 | 42 | FRKLFRVYS | 46 |
| 124 | | | | | |
| SEQ ID NO: | FRKLFHVYS | 0 | 41 | FRKLFRVYS | 46 |
| 125 | | | | | |
| SEQ ID NO: | FRKLFRVYT | 0 | 43 | FRKLFRVYS | 46 |
| 126 | | | | | |
| SEQ ID NO: | FRKLFRVYN | 0 | 43 | FRKLFRVYS | 46 |
| 127 | | | | | |
| SEQ ID NO: | FRKLFRVYD | 0 | 42 | FRKLFRVYS | 46 |
| 128 | | | | | |
| SEQ ID NO: | MRKVFRVYS | 0 | 37 | FRKLFRVYS | 46 |
| 129 | | | | | |
| SEQ ID NO: | MRKLFRTYS | 0 | 37 | FRKLFRVYS | 46 |
| 130 | | | | | |
| SEQ ID NO: | MRKLFRVYA | 0 | 37 | FRKLFRVYS | 46 |
| 131 | | | | | |
| SEQ ID NO: | IRKVFRVYS | 0 | 37 | FRKLFRVYS | 46 |
| 132 | | | | | |
| SEQ ID NO: | IRKLFRTYS | 0 | 37 | FRKLFRVYS | 46 |
| 133 | | | | | |
| SEQ ID NO: | IRKLFRVYA | 0 | 37 | FRKLFRVYS | 46 |
| 134 | | | | | |
| SEQ ID NO: | LRKVFRVYS | 0 | 37 | FRKLFRVYS | 46 |
| 135 | | | | | |
| SEQ ID NO: | LRKLFRTYS | 0 | 37 | FRKLFRVYS | 46 |
| 136 | | | | | |
| SEQ ID NO: | LRKLFRVYA | 0 | 37 | FRKLFRVYS | 46 |
| 137 | | | | | |
| SEQ ID NO: | FRKMFRVYK | 0 | 40 | FRKLFRVYS | 46 |
| 138 | | | | | |
| SEQ ID NO: | FRKIFRVYG | 0 | 40 | FRKLFRVYS | 46 |
| 139 | | | | | |
| SEQ ID NO: | FRKIFRVYE | 0 | 40 | FRKLFRVYS | 46 |
| 140 | | | | | |
| SEQ ID NO: | FRKIFRVYK | 0 | 40 | FRKLFRVYS | 46 |
| 141 | | | | | |
| SEQ ID NO: | FRKVFRTYS | 0 | 40 | FRKLFRVYS | 46 |
| 142 | | | | | |
| SEQ ID NO: | FRKVFRVYA | 0 | 40 | FRKLFRVYS | 46 |
| 143 | | | | | |
| SEQ ID NO: | FRKVFRVYG | 0 | 39 | FRKLFRVYS | 46 |
| 144 | | | | | |
| SEQ ID NO: | FRKVFRVYE | 0 | 39 | FRKLFRVYS | 46 |
| 145 | | | | | |
| SEQ ID NO: | FRKVFRVYK | 0 | 39 | FRKLFRVYS | 46 |
| 146 | | | | | |
| SEQ ID NO: | FRKFFRVYE | 0 | 38 | FRKLFRVYS | 46 |
| 147 | | | | | |
| SEQ ID NO: | FRKFFRVYK | 0 | 38 | FRKLFRVYS | 46 |
| 148 | | | | | |
| SEQ ID NO: | FRKLFRTYA | 0 | 40 | FRKLFRVYS | 46 |
| 149 | | | | | |
| SEQ ID NO: | FRKLFRTYG | 0 | 39 | FRKLFRVYS | 46 |
| 150 | | | | | |
| SEQ ID NO: | FRKLFRTYE | 0 | 39 | FRKLFRVYS | 46 |
| 151 | | | | | |
| SEQ ID NO: | FRKLFRTYK | 0 | 39 | FRKLFRVYS | 46 |
| 152 | | | | | |
| SEQ ID NO: | FRKIFRTYQ | 0 | 37 | FRKLFRVYS | 46 |
| 153 | | | | | |

TABLE 7

Suitable less immunogenic variants of agretope
14 (residues 141-149).

| | | | | | |
|---|---|---|---|---|---|
| | | | | WT sequence | |
| | Variant | I | B | of SEQ ID | B |
| Sequence ID | sequence | (alt) | (alt) | NO: 1 | (wt) |

| | | | | | |
|---|---|---|---|---|---|
| SEQ ID NO: | LWRVYSNFL | 0 | 41 | LFRVYSNFL | 46 |
| 154 | | | | | |
| SEQ ID NO: | LFEVYSNFL | 0 | 41 | LFRVYSNFL | 46 |
| 155 | | | | | |
| SEQ ID NO: | LFQVYSNFL | 0 | 42 | LFRVYSNFL | 46 |
| 156 | | | | | |
| SEQ ID NO: | LFHVYSNFL | 0 | 41 | LFRVYSNFL | 46 |
| 157 | | | | | |
| SEQ ID NO: | LFKVYSNFL | 0 | 43 | LFRVYSNFL | 46 |
| 158 | | | | | |
| SEQ ID NO: | LFRAYSNFL | 0 | 43 | LFRVYSNFL | 46 |
| 159 | | | | | |
| SEQ ID NO: | LFRLYSNFL | 0 | 43 | LFRVYSNFL | 46 |
| 160 | | | | | |
| SEQ ID NO: | LFRVYTNFL | 0 | 43 | LFRVYSNFL | 46 |
| 161 | | | | | |
| SEQ ID NO: | LFRVYANFL | 0 | 43 | LFRVYSNFL | 46 |
| 162 | | | | | |
| SEQ ID NO: | LFRVYGNFL | 0 | 42 | LFRVYSNFL | 46 |
| 163 | | | | | |
| SEQ ID NO: | LFRVYNNFL | 0 | 43 | LFRVYSNFL | 46 |
| 164 | | | | | |
| SEQ ID NO: | LFRVYDNFL | 0 | 42 | LFRVYSNFL | 46 |
| 165 | | | | | |
| SEQ ID NO: | LFRVYENFL | 0 | 42 | LFRVYSNFL | 46 |
| 166 | | | | | |
| SEQ ID NO: | LFRVYQNFL | 0 | 42 | LFRVYSNFL | 46 |
| 167 | | | | | |
| SEQ ID NO: | LFRVYKNFL | 0 | 42 | LFRVYSNFL | 46 |
| 168 | | | | | |
| SEQ ID NO: | LFRVYSSFL | 0 | 41 | LFRVYSNFL | 46 |
| 169 | | | | | |
| SEQ ID NO: | LFRVYSTFL | 0 | 40 | LFRVYSNFL | 46 |
| 170 | | | | | |
| SEQ ID NO: | LFRVYSGFL | 0 | 40 | LFRVYSNFL | 46 |
| 171 | | | | | |
| SEQ ID NO: | LFRVYSDFL | 0 | 41 | LFRVYSNFL | 46 |
| 172 | | | | | |
| SEQ ID NO: | LFRVYSEFL | 0 | 40 | LFRVYSNFL | 46 |
| 173 | | | | | |
| SEQ ID NO: | LFRVYSQFL | 0 | 40 | LFRVYSNFL | 46 |
| 174 | | | | | |
| SEQ ID NO: | LFRVYSHFL | 0 | 41 | LFRVYSNFL | 46 |
| 175 | | | | | |
| SEQ ID NO: | LFRVYSRFL | 0 | 40 | LFRVYSNFL | 46 |
| 176 | | | | | |
| SEQ ID NO: | LFRVYSKFL | 0 | 40 | LFRVYSNFL | 46 |
| 177 | | | | | |
| SEQ ID NO: | LFRVYSNFM | 0 | 44 | LFRVYSNFL | 46 |
| 178 | | | | | |
| SEQ ID NO: | LFRVYSNFV | 0 | 43 | LFRVYSNFL | 46 |
| 179 | | | | | |
| SEQ ID NO: | LFRVYSNFF | 0 | 42 | LFRVYSNFL | 46 |
| 180 | | | | | |

TABLE 8

Suitable less immunogenic variants of agretope
15 (residues 142-150).

| | | | | | |
|---|---|---|---|---|---|
| | | | | WT sequence | |
| | Variant | I | B | of SEQ ID | B |
| Sequence ID | sequence | (alt) | (alt) | NO: 1 | (wt) |

| | | | | | |
|---|---|---|---|---|---|
| SEQ ID NO: | FEVYSNFLR | 0 | 42 | FRVYSNFLR | 47 |
| 181 | | | | | |
| SEQ ID NO: | FRVYSEFLR | 0 | 41 | FRVYSNFLR | 47 |
| 182 | | | | | |
| SEQ ID NO: | FNTYSNFLR | 0 | 39 | FRVYSNFLR | 47 |
| 183 | | | | | |
| SEQ ID NO: | FNAYSNFLR | 0 | 39 | FRVYSNFLR | 47 |
| 184 | | | | | |
| SEQ ID NO: | FNVYSNYLR | 0 | 39 | FRVYSNFLR | 47 |
| 185 | | | | | |
| SEQ ID NO: | FNVYSNFLQ | 0 | 38 | FRVYSNFLR | 47 |
| 186 | | | | | |
| SEQ ID NO: | FNVYSNFLK | 0 | 39 | FRVYSNFLR | 47 |
| 187 | | | | | |
| SEQ ID NO: | FQVHSNFLR | 0 | 38 | FRVYSNFLR | 47 |
| 188 | | | | | |
| SEQ ID NO: | FQVWSNFLR | 0 | 38 | FRVYSNFLR | 47 |
| 189 | | | | | |
| SEQ ID NO: | FQVYSDFLR | 0 | 38 | FRVYSNFLR | 47 |
| 190 | | | | | |
| SEQ ID NO: | FQVYSHFLR | 0 | 38 | FRVYSNFLR | 47 |
| 191 | | | | | |
| SEQ ID NO: | FQVYSNYLR | 0 | 40 | FRVYSNFLR | 47 |
| 192 | | | | | |
| SEQ ID NO: | FQVYSNWLR | 0 | 38 | FRVYSNFLR | 47 |
| 193 | | | | | |
| SEQ ID NO: | FQVYSNFLE | 0 | 38 | FRVYSNFLR | 47 |
| 194 | | | | | |
| SEQ ID NO: | FQVYSNFLK | 0 | 40 | FRVYSNFLR | 47 |
| 195 | | | | | |
| SEQ ID NO: | FHTYSNFLR | 0 | 39 | FRVYSNFLR | 47 |
| 196 | | | | | |
| SEQ ID NO: | FHAYSNFLR | 0 | 39 | FRVYSNFLR | 47 |
| 197 | | | | | |
| SEQ ID NO: | FHVYSNYLR | 0 | 39 | FRVYSNFLR | 47 |
| 198 | | | | | |
| SEQ ID NO: | FHVYSNFLQ | 0 | 38 | FRVYSNFLR | 47 |
| 199 | | | | | |
| SEQ ID NO: | FHVYSNFLK | 0 | 39 | FRVYSNFLR | 47 |
| 200 | | | | | |
| SEQ ID NO: | FKTYSNFLR | 0 | 41 | FRVYSNFLR | 47 |
| 201 | | | | | |
| SEQ ID NO: | FKAYSNFLR | 0 | 41 | FRVYSNFLR | 47 |
| 202 | | | | | |
| SEQ ID NO: | FKVHSNFLR | 0 | 39 | FRVYSNFLR | 47 |
| 203 | | | | | |
| SEQ ID NO: | FKVWSNFLR | 0 | 39 | FRVYSNFLR | 47 |
| 204 | | | | | |
| SEQ ID NO: | FKVYSDFLR | 0 | 39 | FRVYSNFLR | 47 |
| 205 | | | | | |
| SEQ ID NO: | FKVYSQFLR | 0 | 38 | FRVYSNFLR | 47 |
| 206 | | | | | |
| SEQ ID NO: | FKVYSHFLR | 0 | 39 | FRVYSNFLR | 47 |
| 207 | | | | | |
| SEQ ID NO: | FKVYSNYLR | 0 | 41 | FRVYSNFLR | 47 |
| 208 | | | | | |
| SEQ ID NO: | FKVYSNWLR | 0 | 39 | FRVYSNFLR | 47 |
| 209 | | | | | |
| SEQ ID NO: | FKVYSNFLE | 0 | 39 | FRVYSNFLR | 47 |
| 210 | | | | | |
| SEQ ID NO: | FKVYSNFLQ | 0 | 40 | FRVYSNFLR | 47 |
| 211 | | | | | |
| SEQ ID NO: | FKVYSNFLK | 0 | 41 | FRVYSNFLR | 47 |
| 212 | | | | | |
| SEQ ID NO: | FRTWSNFLR | 0 | 39 | FRVYSNFLR | 47 |
| 213 | | | | | |
| SEQ ID NO: | FRTYSDFLR | 0 | 39 | FRVYSNFLR | 47 |
| 214 | | | | | |
| SEQ ID NO: | FRTYSQFLR | 0 | 38 | FRVYSNFLR | 47 |
| 215 | | | | | |
| SEQ ID NO: | FRTYSHFLR | 0 | 39 | FRVYSNFLR | 47 |
| 216 | | | | | |
| SEQ ID NO: | FRAWSNFLR | 0 | 39 | FRVYSNFLR | 47 |
| 217 | | | | | |
| SEQ ID NO: | FRAYSDFLR | 0 | 39 | FRVYSNFLR | 47 |
| 218 | | | | | |
| SEQ ID NO: | FRAYSQFLR | 0 | 38 | FRVYSNFLR | 47 |
| 219 | | | | | |
| SEQ ID NO: | FRAYSHFLR | 0 | 39 | FRVYSNFLR | 47 |
| 220 | | | | | |
| SEQ ID NO: | FRVYSDYLR | 0 | 39 | FRVYSNFLR | 47 |
| 221 | | | | | |
| SEQ ID NO: | FRVYSDFLQ | 0 | 38 | FRVYSNFLR | 47 |
| 222 | | | | | |
| SEQ ID NO: | FRVYSDFLK | 0 | 39 | FRVYSNFLR | 47 |
| 223 | | | | | |
| SEQ ID NO: | FRVYSQYLR | 0 | 38 | FRVYSNFLR | 47 |
| 224 | | | | | |
| SEQ ID NO: | FRVYSQFLK | 0 | 38 | FRVYSNFLR | 47 |
| 225 | | | | | |
| SEQ ID NO: | FRVYSHYLR | 0 | 39 | FRVYSNFLR | 47 |
| 226 | | | | | |
| SEQ ID NO: | FRVYSHFLK | 0 | 39 | FRVYSNFLR | 47 |
| 227 | | | | | |
| SEQ ID NO: | FRTYSNYLK | 0 | 38 | FRVYSNFLR | 47 |
| 228 | | | | | |
| SEQ ID NO: | FRAYSNYLK | 0 | 38 | FRVYSNFLR | 47 |
| 229 | | | | | |

TABLE 9

Suitable less immunogenic variants of agretope
18 (residues 149-157).

| | | | | WT sequence | |
|---|---|---|---|---|---|
| | Variant | I | B | of SEQ ID | B |
| Sequence ID | sequence | (alt) | (alt) | NO: 1 | (wt) |
| SEQ ID NO: | LNGKLKLYT | 0 | 40 | LRGKLKLYT | 45 |
| 230 | | | | | |
| SEQ ID NO: | LEGKLKLYT | 0 | 40 | LRGKLKLYT | 45 |
| 231 | | | | | |
| SEQ ID NO: | LQGKLKLYT | 0 | 41 | LRGKLKLYT | 45 |
| 232 | | | | | |
| SEQ ID NO: | LHGKLKLYT | 0 | 40 | LRGKLKLYT | 45 |
| 233 | | | | | |
| SEQ ID NO: | LKGKLKLYT | 0 | 42 | LRGKLKLYT | 45 |
| 234 | | | | | |
| SEQ ID NO: | LRGSLKLYT | 0 | 40 | LRGKLKLYT | 45 |
| 235 | | | | | |
| SEQ ID NO: | LRGELKLYT | 0 | 41 | LRGKLKLYT | 45 |
| 236 | | | | | |
| SEQ ID NO: | LRGQLKLYT | 0 | 41 | LRGKLKLYT | 45 |
| 237 | | | | | |
| SEQ ID NO: | LRGKLSLYT | 0 | 40 | LRGKLKLYT | 45 |
| 238 | | | | | |
| SEQ ID NO: | LRGKLNLYT | 0 | 40 | LRGKLKLYT | 45 |
| 239 | | | | | |
| SEQ ID NO: | LRGKLELYT | 0 | 41 | LRGKLKLYT | 45 |
| 240 | | | | | |
| SEQ ID NO: | LRGKLQLYT | 0 | 41 | LRGKLKLYT | 45 |
| 241 | | | | | |
| SEQ ID NO: | LRGKLKIYT | 0 | 43 | LRGKLKLYT | 45 |
| 242 | | | | | |
| SEQ ID NO: | LRGKLKVYT | 0 | 42 | LRGKLKLYT | 45 |
| 243 | | | | | |
| SEQ ID NO: | LRAKLRLYT | 0 | 36 | LRGKLKLYT | 45 |
| 244 | | | | | |
| SEQ ID NO: | LRGNLRLYT | 0 | 37 | LRGKLKLYT | 45 |
| 245 | | | | | |
| SEQ ID NO: | LRGNLKMYT | 0 | 38 | LRGKLKLYT | 45 |
| 246 | | | | | |
| SEQ ID NO: | LRGNLKFYT | 0 | 36 | LRGKLKLYT | 45 |
| 247 | | | | | |
| SEQ ID NO: | LRGRLKFYT | 0 | 38 | LRGKLKLYT | 45 |
| 248 | | | | | |

### Example 3

### Identification of Suitable Less Immunogenic Sequences for MHC-binding Agretopes in Erythropoietin as Determined by PDA® Technology

Each position in the agretopes of interest was analyzed to identify a subset of amino acid substitutions that are potentially compatible with maintaining the structure and function of the protein. PDA® technology calculations were run for each position of each nine-mer agretope and compatible amino acids for each position were saved. In these calculations, side-chains within 5 Angstroms of the position of interest were permitted to change conformation but not amino acid identity. The variant agretopes were then analyzed for immunogenicity. The PDA® energies and Iscore values for the wild-type nine-mer agretope were compared to the variants and the subset of variant sequences with lower predicted immunogenicity and PDA® energies within 5.0 kcal/mol of the wild-type (wt) were noted. In Tables 10-15, E(PDA) is the energy determined using PDA® technology calculations compared against the wild-type, Iscore: Anchor is the Iscore for the agretope, and Iscore: Overlap is the sum of the Iscores for all of the overlapping agretopes.

TABLE 10
Suitable less immunogenic variants of agretope 3 (residues 51-59).

| | | Iscore: | Iscore: |
|---|---|---|---|
| Var. | E (PDA) | Anchor | Overlap |
| wt | 0 | 34.03 | 5.87 |
| K52E | 2.28 | 14.43 | 5.87 |
| K52D | 3.70 | 1.25 | 5.87 |
| R53Q | 0.68 | 22.25 | 0.00 |
| R53N | 0.92 | 27.17 | 1.92 |
| R53H | 1.36 | 26.56 | 0.00 |
| R53S | 1.59 | 26.56 | 1.92 |
| R53E | 1.83 | 6.40 | 0.00 |
| RS3A | 1.91 | 22.25 | 0.49 |
| R53D | 2.68 | 6.03 | 0.00 |
| R53G | 2.71 | 26.56 | 1.23 |
| M54K | 1.11 | 12.90 | 2.19 |
| M54Q | 1.21 | 16.09 | 0.00 |
| MS4T | 1.54 | 11.01 | 0.00 |

TABLE 11
Suitable less immunogenic variants of agretope 11 (residues 102-110).

| | | Iscore: | Iscore: |
|---|---|---|---|
| Var. | E (PDA) | Anchor | Overlap |
| wt | 0 | 63.06 | 2.31 |
| R103K | 0.76 | 21.86 | 2.31 |
| R1031 | 3.83 | 21.86 | 2.31 |
| R103H | 4.36 | 13.43 | 2.31 |
| R103M | 4.63 | 21.86 | 2.31 |
| S104A | 0.52 | 49.66 | 2.31 |
| S104T | 3.61 | 49.66 | 2.31 |
| L105I | 0.50 | 56.30 | 2.31 |
| L105V | 2.93 | 32.04 | 2.31 |
| T107K | −3.51 | 47.67 | 2.31 |
| T107R | −1.27 | 51.47 | 2.31 |
| T107N | −0.57 | 33.29 | 2.31 |
| T107G | 0.85 | 25.58 | 2.31 |
| T107D | 1.53 | 0.00 | 2.31 |
| T107E | 1.57 | 1.11 | 2.31 |
| L108E | 3.45 | 18.11 | 2.31 |
| L108Q | 5.00 | 27.59 | 2.31 |
| R110K | 1.44 | 48.64 | 0.00 |
| R110N | 1.82 | 38.57 | 0.00 |
| R110H | 1.85 | 50.60 | 0.00 |
| R110Q | 2.34 | 51.12 | 0.00 |
| R110T | 2.67 | 52.87 | 0.00 |
| R110D | 2.87 | 32.43 | 0.00 |
| R110Y | 3.22 | 59.98 | 0.00 |

TABLE 12
Suitable less immunogenic variants of agretope 13 (residues 138-146).

| | | Iscore: | Iscore: |
|---|---|---|---|
| Var. | E (PDA) | Anchor | Overlap |
| wt | 0 | 42.21 | 68.53 |
| R139H | −1.15 | 11.86 | 68.53 |
| R139K | −0.76 | 11.86 | 68.53 |
| R139P | −0.74 | 0.00 | 68.53 |
| R139Q | −0.40 | 11.86 | 68.53 |
| R139N | 0.31 | 11.86 | 68.53 |
| R139G | 1.84 | 8.94 | 68.53 |
| K140D | 1.03 | 11.86 | 68.53 |
| K140E | 3.84 | 11.86 | 68.53 |
| L141K | 2.85 | 28.32 | 35.92 |
| L141I | 3.71 | 37.06 | 68.53 |
| L141Q | 4.62 | 28.69 | 35.92 |
| L141V | 4.64 | 24.83 | 68.53 |
| R143M | −0.86 | 18.60 | 56.85 |
| R143L | −0.86 | 34.59 | 48.92 |
| R143K | −0.68 | 41.58 | 24.71 |
| R143H | 0.63 | 20.90 | 25.79 |
| R143Q | 0.77 | 20.57 | 34.52 |
| R143E | 1.31 | 5.48 | 8.94 |
| R143W | 1.66 | 14.18 | 27.01 |
| R143D | 2.87 | 4.52 | 1.65 |
| R143G | 3.44 | 28.02 | 19.13 |
| V144K | −0.51 | 35.69 | 21.63 |
| V144T | 1.76 | 27.74 | 51.98 |
| V144N | 1.97 | 36.61 | 36.73 |
| R139T | −0.30 | 2.80 | 68.53 |
| R139E | −0.26 | 2.80 | 68.53 |
| R139D | 0.00 | 0.00 | 68.53 |
| R139S | 0.28 | 0.00 | 68.53 |
| R139A | 0.29 | 2.80 | 68.53 |
| V144E | 2.86 | 20.06 | 5.90 |
| V144A | 2.93 | 40.57 | 38.77 |
| V144Q | 2.95 | 35.10 | 24.24 |
| V144H | 3.74 | 36.44 | 50.07 |
| V144S | 3.91 | 30.59 | 51.98 |
| V144D | 4.32 | 8.02 | 10.39 |
| S146F | −5.89 | 26.33 | 57.42 |
| S146L | −4.21 | 31.83 | 59.95 |
| S146Y | −3.89 | 20.64 | 57.42 |
| S146E | −3.30 | 13.86 | 35.50 |
| S146W | −3.19 | 8.11 | 41.57 |
| S146H | −3.06 | 27.46 | 56.79 |
| S146D | −0.89 | 7.63 | 34.28 |
| S146A | −0.81 | 27.67 | 55.17 |
| S146M | −0.81 | 33.78 | 64.71 |
| S146K | −0.67 | 18.86 | 57.33 |
| S146Q | 0.01 | 27.03 | 57.33 |
| S146T | 1.08 | 18.80 | 54.73 |
| S146G | 1.92 | 25.96 | 60.09 |

TABLE 13
Suitable less immunogenic variants of agretope 14 (residues 141-149).

| | | Iscore: | Iscore: |
|---|---|---|---|
| Var. | E (PDA) | Anchor | Overlap |
| wt | 0 | 32.60 | 110.14 |
| L141K | 2.85 | 0.00 | 96.24 |
| L141Q | 4.62 | 0.00 | 96.61 |
| R143K | −0.68 | 15.17 | 83.12 |
| R143T | 0.11 | 15.17 | 95.45 |
| R143A | 0.42 | 15.17 | 86.78 |
| R143H | 0.63 | 16.25 | 62.43 |
| R143Q | 0.77 | 15.17 | 71.92 |
| R143N | 1.03 | 31.43 | 89.07 |
| R143E | 1.31 | 6.07 | 40.36 |
| R143S | 1.61 | 16.25 | 83.44 |
| R143W | 1.66 | 15.17 | 58.02 |
| R143D | 2.87 | 0.00 | 38.17 |
| R143G | 3.44 | 16.25 | 62.90 |
| V144K | −0.51 | 0.00 | 89.32 |
| V144T | 1.76 | 30.34 | 81.38 |
| V144N | 1.97 | 1.23 | 104.12 |
| V144E | 2.86 | 3.44 | 54.52 |
| V144A | 2.93 | 17.14 | 94.20 |
| V144Q | 2.95 | 2.60 | 88.73 |
| V144H | 3.74 | 28.44 | 90.07 |
| V144S | 3.91 | 30.34 | 84.23 |
| V144D | 4.32 | 7.94 | 42.48 |
| S146F | −5.89 | 6.07 | 109.68 |
| S146Y | −3.89 | 6.07 | 103.99 |
| S146E | −3.30 | 0.00 | 81.36 |
| S146W | −3.19 | 6.07 | 75.61 |
| S146N | −1.53 | 18.42 | 92.67 |
| S146D | −0.89 | 0.00 | 73.91 |
| S146A | −0.81 | 19.67 | 95.17 |
| S146K | −0.67 | 6.07 | 102.13 |
| S146T | 1.08 | 19.23 | 86.30 |
| S146G | 1.92 | 17.51 | 100.54 |
| N147D | 3.11 | 0.00 | 90.25 |
| L149T | 3.62 | 1.45 | 80.85 |
| L149N | 4.73 | 2.53 | 78.76 |
| L149D | 4.95 | 1.11 | 77.71 |

TABLE 14
Suitable less immunogenic variants of agretope 15 (residues 142-150).

| | | Iscore: | Iscore: |
|---|---|---|---|
| Var. | E (PDA) | Anchor | Overlap |
| wt | 0 | 34.28 | 108.46 |
| F142H | 4.04 | 0.00 | 108.46 |
| R143M | −0.86 | 7.89 | 99.57 |
| R143L | −0.86 | 7.89 | 107.63 |
| R143K | −0.68 | 7.89 | 90.41 |
| R143A | 0.42 | 1.23 | 100.72 |
| R143H | 0.63 | 7.89 | 70.80 |
| R143Q | 0.77 | 17.70 | 69.39 |
| R143E | 1.31 | 1.23 | 45.20 |
| R143S | 1.61 | 1.23 | 98.46 |
| R143W | 1.66 | 1.23 | 71.97 |
| R143D | 2.87 | 0.00 | 38.17 |
| R143G | 3.44 | 1.23 | 77.92 |
| V144K | −0.51 | 20.41 | 68.92 |
| V144T | 1.76 | 20.41 | 91.31 |
| V144E | 2.86 | 1.23 | 56.73 |
| V144A | 2.93 | 20.41 | 90.93 |
| V144Q | 2.95 | 20.41 | 70.93 |
| V144H | 3.74 | 20.41 | 98.11 |
| V144S | 3.91 | 20.41 | 94.16 |
| V144D | 4.32 | 1.23 | 49.19 |
| Y145W | 1.28 | 14.95 | 108.05 |
| Y145K | 4.88 | 11.30 | 107.24 |
| N147D | 3.11 | 16.03 | 74.21 |
| F148H | 4.76 | 20.41 | 107.24 |
| R150K | 1.85 | 19.52 | 80.75 |
| R150E | 4.00 | 25.09 | 74.82 |
| R150Q | 4.23 | 29.41 | 81.68 |

TABLE 15
Suitable less immunogenic variants of agretope 18 (residues 149-157).

| | | Iscore: | Iscore: |
|---|---|---|---|
| Var. | E (PDA) | Anchor | Overlap |
| wt | 0 | 32.00 | 82.78 |
| L149T | 3.62 | 0.00 | 54.34 |
| L149N | 4.73 | 0.00 | 53.33 |
| L149D | 4.95 | 0.00 | 50.86 |
| R150K | 1.85 | 5.93 | 66.38 |
| R150E | 4.00 | 0.00 | 71.94 |
| R150Q | 4.23 | 6.87 | 76.27 |
| G151A | 3.71 | 21.98 | 82.78 |
| K152P | −0.58 | 0.00 | 82.36 |
| K154F | −0.54 | 0.00 | 74.08 |
| K154W | −0.36 | 0.00 | 68.53 |
| K154L | 0.88 | 5.93 | 82.78 |
| K154M | 0.96 | 0.00 | 82.78 |
| K154A | 1.39 | 5.93 | 68.53 |
| K154H | 1.49 | 1.65 | 74.08 |
| K154T | 1.84 | 18.02 | 68.53 |
| K154Y | 2.26 | 5.55 | 82.78 |
| K154N | 2.45 | 6.50 | 74.08 |
| K154S | 2.67 | 7.75 | 68.53 |
| K154Q | 2.87 | 1.25 | 82.78 |
| K154D | 2.91 | 0.00 | 68.53 |
| K154E | 4.04 | 0.00 | 68.53 |
| K154G | 4.42 | 5.55 | 74.08 |
| L155E | 3.08 | 0.42 | 68.53 |
| L155N | 3.28 | 14.68 | 74.08 |
| L155D | 3.51 | 0.00 | 68.53 |
| L155Q | 4.18 | 11.95 | 68.53 |
| L155W | 4.68 | 22.05 | 68.53 |
| T157N | 1.77 | 28.12 | 82.78 |
| T157D | 2.76 | 28.12 | 82.78 |
| T157E | 4.85 | 28.12 | 82.78 |

The amino acid sequence of a wild type human erythropoietin protein (SEQ ID NO:1), Protein Data Bank 1EER, is shown below.

```
      >EPO (SEQ ID NO: 1)
      APPRLICDSRVLERYLLEAKEAEKITTGCAEHCSLNEKITVPDTKVNFYAWKRMEVGQQAVEVWQ
GLALLSEAVLRGQALLVKSSQPWEPLQLHVDKAVSGLRSLTTLLRALGAQKEAISNSDAASAAPLRTIT
ADTFRKLFRVYSNFLRGKLKLYTGEACRTGDR
```

EPO, as used herein, also refers to wild-type human erythropoietin protein including the following sequence:

EPO (SEQ ID NO:249)

APPRLICDSRVLERYLLEAKEAENITTGCAEHCSLNENITVPDTKVNFYAWKRMEVGQQAVEVW
QGLALLSEAVLRGQALLVNSSQPWEPLQLHVDKAVSGLRSLTTLLRALGAQKEAISPPDAASAAPLRT
ITADTFRKLFRVYSNFLRGKLKLYTGEACRTGDR.

The wild-type human erythropoietin protein also refers to sequences corresponding to SEQ ID NO:1 or 249 that lack the C terminal arginine, sequences including an N-terminal methionine at the -1 position, and sequences substituting methionine for the N-terminal Alanine.

We claim:

1. A non-naturally occurring variant EPO protein having reduced immunogenicity as compared with a naturally occurring EPO protein comprising SEQ ID NO: 1 or SEQ ID NO: 249, wherein said variant protein comprises at least two amino acid modifications as compared to said naturally occurring EPO protein.

2. A variant EPO protein of claim 1, wherein at least one said modification is made to an amino acid in an agretope selected from the group consisting of: agretope 1: residues 5-13; agretope 2: residues 48-56; agretope 3: residues 51-59; agretope 4: residues 61-69; agretope 5: residues 64-72; agretope 6: residues 69-77; agretope 7: residues 74-82; agretope 8: residues 75-83; agretope 9: residues 80-88; agretope 10: residues 93-101; agretope 11: residues 102-110; agretope 12: residues 109-117; agretope 13: residues 138-146; agretope 14: residues 141-149; agretope 15: residues 142-150; agretope 16: residues 144-152; agretope 17: residues 145-153; agretope 18: residues 149-157; agretope 19: residues 153-161; and agretope 20: residues 156-164.

3. A variant EPO protein of claim 1, wherein at least one amino acid modification is made to an amino acid in agretope 1 (residues 5-13).

4. A variant EPO protein of claim 1, wherein at least one amino acid modification is made to an amino acid in agretope 3 (residues 51-59).

5. A variant EPO protein of claim 1, wherein at least one amino acid modification is made to an amino acid in agretope 5 (residues 64-72).

6. A variant EPO protein of claim 1, wherein at least one amino acid modification is made to an amino acid in agretope 10 (residues 93-101).

7. A variant EPO protein of claim 1, wherein at least one amino acid modification is made to an amino acid in agretope 11 (residues 102-110).

8. A variant EPO protein of claim 1, wherein at least one amino acid modification is made to an amino acid in agretope 13 (residues 138-146).

9. A variant EPO protein of claim 1, wherein at least one amino acid modification is made to an amino acid in agretope 14 (residues 141-149).

10. A variant EPO protein of claim 1, wherein at least one amino acid modification is made to an amino acid in agretope 15 (residues 142-150).

11. A variant EPO protein of claim 1, wherein at least one amino acid modification is made to agretope 18 (residues 149-157).

12. A variant EPO protein of claim 1, wherein at least one amino acid modification is made to an amino acid in agretope 19 (residues 153-161).

13. A variant EPO protein of claim 1, wherein at least one modification is to an amino acid at a position selected from an the group consisting of positions 52, 53, 54, 56, 57, 59, 103, 104, 105, 107, 108, 110, 139, 140, 141, 142, 143, 144, 145, 146, 147, 148, 149, 150, 151, 152, 154, 155, and 157; and wherein: modifications to position 52 are selected from the group consisting of D and E; modifications to position 53 are selected from the group consisting of A, D, E, G, H, N, Q, and S; modifications to position 54 are selected from the group consisting of A, D, E, G, H, K, Q, S, T, V, W, and Y; modifications to position 56 are selected from the group consisting of D, E, L, N, P, W, and Y; modifications to position 57 are selected from the group consisting of D and E; modifications to position 59 are selected from the group consisting of A, E, F, H, l, K, L, M, N, R, W, and Y; modifications to position 103 are selected from the group consisting of H, l, K, and M; modifications to position 104 are selected from the group consisting of A and T; modifications to position 105 are selected from the group consisting of I and V; modifications to position 107 are selected from the group consisting of D, E, G, K, N, and R; modifications to position 108 are selected from the group consisting of E and Q; modifications to position 110 are selected from the group consisting of D, E, G, H, K, N, Q, T, and Y; modifications to position 139 are selected from the group consisting of A, D, E, G, H, K, N, P, Q, S, and T; modifications to position 140 are selected from the group consisting of D and E; modifications to position 141 are selected from the group consisting of I, K, Q, and V; modifications to position 142 is H; modifications to position 143 are selected from the group consisting of A, D, E, G, H, K, L, M, N, Q, S, T, and W; modifications to position 144 are selected from the group consisting of A, D, E, H, K, N, Q, S, and T; modifications to position 145 are selected from the group consisting of K and W; modifications to position 146 are selected from the group consisting of A, D, E, F, G, H, K, L, M, N, Q, T, W, and Y; the modification to position 147 is D; modification to position 148 is H; modifications to position 149 are selected from the group consisting of D, N, and T; modifications to position 150 are selected from the group consisting of E, K, and Q; the modification to position 151 is A; the modification to position 152 is P; modifications to position 154 are selected from the group consisting of A, D, E, F, G, H, L, M, N, Q, S, T, W, and Y; modifications to position 155 are selected from the group consisting of D, E, N, Q, and W; and modifications to position 157 are selected from the group consisting of D, E, and N.

14. A composition comprising a variant human EPO monomer comprising the formula:
Fx(1-51)-Vb(52)-Vb(53)-Vb(54)-Fx(55)-Vb(56)-
Vb(57)-Fx(58)-Vb(59)-Fx(60-102)-Vb(103)-Vb(104)-
Vb(105)-Fx(106)-Vb(107)-Vb(108)-Fx(109)-Vb(110)-
Fx(111-138)-Vb(139)-Vb(140)-Vb(141)-Vb(142)-
Vb(143)-Vb(144)-Vb(145)-Vb(146)-Vb(147)-Vb(148)-
Vb(149)-Vb(150)-Vb(151)-Vb(152)-Fx(153)-Vb(154)-
Vb(155)-Fx(156)-Vb(157)-Fx(158-165)
wherein:
Fx(1-51) comprises the amino acid sequence of SEQ ID NO: 1 or SEQ ID NO: 249 at positions 1-51;
Vb(52) is selected from the group consisting of D, E, and K;
Vb(53) is selected from the group consisting of A, D, E, G, H, N, Q, R, and S;
Vb(54) is selected from the group consisting of A, D, E, G, H, K, M, Q, S, T, V, W, and Y;
Fx(55) comprises the amino acid sequence of SEQ ID NO: 1 or SEQ ID NO: 249 at position 55;
Vb(56) is selected from the group consisting of D, E, L, N, P, V, W, and Y;
Vb(57) is selected from the group consisting of D, E, and G;
Fx(58) comprises the amino acid sequence of SEQ ID NO: 1 or SEQ ID NO: 249 at position 58;
Vb(59) is selected from the group consisting of A, E, F, H, I, K, L, M, N, Q, R, W, and Y;
Fx(60-102) comprises the amino acid sequence of SEQ. ID. NO: 1 or SEQ ID NO: 249 at positions 60-102;
Vb(103) is selected from the group consisting of H, I, K, M, and R;
Vb(104) is selected from the group consisting of A, S, and T;
Vb(105) is selected from the group consisting of I, L, and V;
Fx(106) comprises the amino acid sequence of SEQ ID NO: 1 or SEQ ID NO: 249 at position 106;
Vb(107) is selected from the group consisting of D, E, G, K, N, R, and T;
Vb(108) is selected from the group consisting of E, L, and Q;
Fx(109) comprises the amino acid sequence of SEQ ID NO: 1 or SEQ ID NO: 249 at position 109;
Vb(110) is selected from the group consisting of D, E, G, H, K, N, Q, R, T, and Y;
Fx(111-138) comprises the amino acid sequence of SEQ ID NO: 1 or SEQ ID NO: 249 at positions 111-138;
Vb(139) is selected from the group consisting of A, D, E, G, H, K, N, P, Q, R, S, and T;
Vb(140) is selected from the group consisting of D, E, and K;
Vb(141) is selected from the group consisting of 1, K, L, Q, and V;
Vb(142) is selected from the group consisting of F and H;
Vb(143) is selected from the group consisting of A, D, E, G, H, K, L, M, N, Q, R, S, T, and W;
Vb(144) is selected from the group consisting of A, D, E, H, K, N, Q, S, T, and V;
Vb(145) is selected from the group consisting of K, W, and Y;
Vb(146) are selected from the group consisting of A, D, E, F, G, H, K, L, M, N, Q, S, T, W, and Y;
Vb(147) is selected from the group consisting of D and N;
Vb(148) is selected from the group consisting of F and H;
Vb(149) is selected from the group consisting of D, L, N, and T;
Vb(150) is selected from the group consisting of E, K, Q, and R;
Vb(151) is selected from the group consisting of A and G;
Vb(152) is selected from the group consisting of K and P;
Fx(153) comprises the amino acid sequence of SEQ ID NO: 1 or SEQ ID NO: 249 at position 153;
Vb(154) is selected from the group consisting of A, D, E, F, G, H, K, L, M, N, Q, S, T, W, and Y;
Vb(155) is selected from the group consisting of D, E, L, N, Q, and W;
Fx(156) comprises the amino acid sequence of SEQ ID NO: 1 or SEQ ID NO: 249) at position 156;
Vb(157) is selected from the group consisting of D, E, N, and T;
Fx(158-165) comprises the amino acid sequence of SEQ ID NO: 1 or SEQ ID NO: 249 at positions 158-165; and
wherein said variant has at least two amino acid substitutions as compared to SEQ ID NO: 1 or SEQ ID NO: 249.
